SEQUENCE LISTING (8 sequences)

SEQ ID NO: 1 - DNA, 927 bp, synthetic construct
  misc_feature 1..927: note = 30S ribosomal protein
  source 1..927: mol_type = other DNA
atggcaacgcttttaggtttctctcagactaagtttcaccattgcggcgtctggatttctacgccgccgtgctcatcttcttccacggtagtctcaatggtgggcttgaatcggactgattcgaaaaagctccgttcagatttcctcggccagattggttacgaagatcggagacaagtaagacacagttactgtagaagctcattagctgtgaaaatgtcgtgggacggtcctcttgcttccgtcaagttgatcatccaaggaaaaaacctcgagttatcagagccaattaagcagcatgttgaagagaaagtaggcaaatctgttcagaaacacagtcatcttgtgagagaagttgatgtaagactctctgttcgtggtggagagtttggtaaaggccctaggattcgaagatgtgaggtgacattgtttacaaagaagcatggtgttgtgcgtgctgaggaagatgctgagacagtatacgcttgtatcgacttggtatcaacgataatacagaggaagctgaggaagatcaaggagaaggactcagaccatggaaggcacatgaaaggtttcaacagattgaaggtaagggaaccagtgattgagccggttgtggaggatgttgaggacagtactgactcgagcgtaggagaagaagaagaagaggatgatttgatcaaggagattgtccgtaccaagactttcgagatgccaccattgactgtcgctgaggcagtcgagcagctggaactagtcagtcacgacttctatggcttccaaaatgaagaaactggtgagataaacatagtgtacaagagaaaagaaggaggttacggtctgataatcccaaagaaagacgggaaggccgagaaggttgagccgcttccaaccgagcaattgaatgaacactctttcgccgagtag

SEQ ID NO: 2 - protein, 308 aa, synthetic construct
  REGION 1..308: note = 30S ribosomal protein
MATLLGFSQTKFHHCGVWISTPPCSSSSTVVSMVGLNRTDSKKLRSDFLGQIGYEDRRQVRHSYCRSSLAVKMSWDGPLASVKLIIQGKNLELSEPIKQHVEEKVGKSVQKHSHLVREVDVRLSVRGGEFGKGPRIRRCEVTLFTKKHGVVRAEEDAETVYACIDLVSTIIQRKLRKIKEKDSDHGRHMKGFNRLKVREPVIEPVVEDVEDSTDSSVGEEEEEDDLIKEIVRTKTFEMPPLTVAEAVEQLELVSHDFYGFQNEETGEINIVYKRKEGGYGLIIPKKDGKAEKVEPLPTEQLNEHSFAE

SEQ ID NO: 3 - DNA, 2529 bp, synthetic construct
  misc_feature 1..2529: note = CHX20
  source 1..2529: mol_type = other DNA
atgcccttcaacataacctccgtgaaaacctcatctaacggagtatggcaaggcgacaatcctttaaacttcgcttttccgttactcatcgtccaaacggcgttaatcatcgccgtcagtcgcttcctcgccgtcttattcaaacctctccgtcaacccaaagtcatcgccgagattgtcggagggattttgttaggaccatcggctttaggtagaaacatggcgtacatggaccgtatatttccgaaatggagtatgccgatactcgaatccgtcgcgagcataggacttctcttcttcctcttcctcgtcggtctagaactcgatttatcatcgatccgacgaagcggcaaacgcgctttcggaatcgcagtcgctggaattacactaccgtttatcgccggcgtcggagtcgcgtttgtgatccgtaacactctctacaccgccgcggataaaccaggttacgccgagtttctcgttttcatgggagtcgcactctcgatcacagcttttccggtacttgcgcgtattttagcagagctcaagcttttaacgactcagataggagaaaccgcgatggctgcagccgcttttaacgatgtagccgcgtggattttactcgctttagcggttgcgttagcgggtaatggcggtgagggaggtggagagaaaaagagtccgttagtgtcgttgtgggttttgttatcgggcgctgggtttgtggtttttatgttggttgtgatccgacccggaatgaaatgggtcgcgaaacgtggatctcctgaaaacgacgtcgtacgcgagtcttacgtgtgtttgacgttagccggtgttatggtttccggtttcgcgacggatttaattgggattcattcgatttttggagcgtttgttttcggtttgactataccgaaagatggagagtttggtcagcgattgattgaacgaattgaggattttgtttccggtttactcttaccgctttatttcgctacgagtggtttgaagactgacgtggctaagattagaggagctgagtcgtgggggatgttgggtcttgttgttgttacggcttgtgccgggaagatagtcggaacttttgttgtggcggtgatggttaaagttccggcgagagaggcgttgacacttggtttcttgatgaatactaaaggtttagtggagctcattgtactcaacataggcaaggagaaaaaggtactaaacgacgagacgtttgcaatactagtgctaatggcactcttcacaacgttcataacgacgcctactgtaatggccatttacaagccggcacgtggcacccaccgcaaactaaaagacttgtcggcgagccaagactccaccaaggaagagcttcgcatcctagcctgcctccacggcccagccaatgtctcctccctcatctctctcgtcgagtccatccgaaccaccaagatactacggctaaagctgtttgtgatgcatctgatggaactaacggaacggtcttcgtcaatcataatggtgcaaagagcccgtaaaaacggacttcctttcgttcaccgttaccgtcatggtgagcgtcacagcaacgtcataggaggcttcgaagcctatcgtcaactaggccgggtcgcagtccggcccatcaccgcagtctctccattacccacaatgcacgaagacatttgccacatggcagataccaagagggtcacaatgatcattttacctttccacaaacgatggaacgctgatcatggtcatagccaccaccaccaagacggaggaggagatggaaacgtaccggaaaacgttggtcatggttggcgattggttaaccaaagggttttgaagaatgcgccgtgttcggtggcggttcttgtagaccgtggacttgggtccattgaggcccaaactttgagcttagatgggtcgaatgtggttgaaagggtttgtgtgattttctttggtgggcctgatgaccgtgagtctatagagctcggcgggagaatggctgagcatccggccgttaaagttaccgttattaggtttttggtaagagaaacgttgaggagtaccgccgtcactttacgaccggcaccgtctaaaggcaaggagaagaactatgcctttttaacaaccaacgtggatccagaaaaagaaaaggaattagacgaaggggcattggaagacttcaagagcaaatggaaagaaatggtggagtacaaagaaaaggaaccaaacaacatcattgaagaaatactgtcaataggacagagtaaagactttgatctaatagtggttggaagagggaggataccgtcggccgaggtggcggcattagctgagcgtcaagctgaacatcctgagttaggtcctatcggagacgtgctcgcctcttcgatcaaccacatcattccatcaatccttgtggttcaacaacacaacaaagctcatgtagaggatattacggtttccaaaattgttagtgagtcttctctaagtattaacggagacacaaatgtatga

SEQ ID NO: 4 - protein, 842 aa, synthetic construct
  REGION 1..842: note = CHX20
MPFNITSVKTSSNGVWQGDNPLNFAFPLLIVQTALIIAVSRFLAVLFKPLRQPKVIAEIVGGILLGPSALGRNMAYMDRIFPKWSMPILESVASIGLLFFLFLVGLELDLSSIRRSGKRAFGIAVAGITLPFIAGVGVAFVIRNTLYTAADKPGYAEFLVFMGVALSITAFPVLARILAELKLLTTQIGETAMAAAAFNDVAAWILLALAVALAGNGGEGGGEKKSPLVSLWVLLSGAGFVVFMLVVIRPGMKWVAKRGSPENDVVRESYVCLTLAGVMVSGFATDLIGIHSIFGAFVFGLTIPKDGEFGQRLIERIEDFVSGLLLPLYFATSGLKTDVAKIRGAESWGMLGLVVVTACAGKIVGTFVVAVMVKVPAREALTLGFLMNTKGLVELIVLNIGKEKKVLNDETFAILVLMALFTTFITTPTVMAIYKPARGTHRKLKDLSASQDSTKEELRILACLHGPANVSSLISLVESIRTTKILRLKLFVMHLMELTERSSSIIMVQRARKNGLPFVHRYRHGERHSNVIGGFEAYRQLGRVAVRPITAVSPLPTMHEDICHMADTKRVTMIILPFHKRWNADHGHSHHHQDGGGDGNVPENVGHGWRLVNQRVLKNAPCSVAVLVDRGLGSIEAQTLSLDGSNVVERVCVIFFGGPDDRESIELGGRMAEHPAVKVTVIRFLVRETLRSTAVTLRPAPSKGKEKNYAFLTTNVDPEKEKELDEGALEDFKSKWKEMVEYKEKEPNNIIEEILSIGQSKDFDLIVVGRGRIPSAEVAALAERQAEHPELGPIGDVLASSINHIIPSILVVQQHNKAHVEDITVSKIVSESSLSINGDTNV

SEQ ID NO: 5 - DNA, 39 bp, synthetic construct
  misc_feature 1..39: note = At30S-gib-Forward
  source 1..39: mol_type = other DNA
tctatctctctcgaccgctatggcaacgcttttaggttt

SEQ ID NO: 6 - DNA, 40 bp, synthetic construct
  misc_feature 1..40: note = At30S-gib-Reverse
  source 1..40: mol_type = other DNA
tcgaaccactttgtacaagactcggcgaaagagtgttcat

SEQ ID NO: 7 - DNA, 1676 bp, synthetic construct
  misc_feature 1..1676: note = 30S sequence attached with GFP
  source 1..1676: mol_type = other DNA
atggcaacgcttttaggtttctctcagactaagtttcaccattgcggcgtctggatttctacgccgccgtgctcatcttcttccacggtagtctcaatggtgggcttgaatcggactgattcgaaaaagctccgttcagatttcctcggccagattggttacgaagatcggagacaagtaagacacagttactgtagaagctcattagctgtgaaaatgtcgtgggacggtcctcttgcttccgtcaagttgatcatccaaggaaaaaacctcgagttatcagagccaattaagcagcatgttgaagagaaagtaggcaaatctgttcagaaacacagtcatcttgtgagagaagttgatgtaagactctctgttcgtggtggagagtttggtaaaggccctaggattcgaagatgtgaggtgacattgtttacaaagaagcatggtgttgtgcgtgctgaggaagatgctgagacagtatacgcttgtatcgacttggtatcaacgataatacagaggaagctgaggaagatcaaggagaaggactcagaccatggaaggcacatgaaaggtttcaacagattgaaggtaagggaaccagtgattgagccggttgtggaggatgttgaggacagtactgactcgagcgtaggagaagaagaagaagaggatgatttgatcaaggagattgtccgtaccaagactttcgagatgccaccattgactgtcgctgaggcagtcgagcagctggaactagtcagtcacgacttctatggcttccaaaatgaagaaactggtgagataaacatagtgtacaagagaaaagaaggaggttacggtctgataatcccaaagaaagacgggaaggccgagaaggttgagccgcttccaaccgagcaattgaatgaacactctttcgccgagtctagtacaaagtggttcgatctagaggatccatggtgagcaagggcgaggagctgttcaccggggtggtgcccatcctggtcgagctggacggcgacgtgaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaa

SEQ ID NO: 8 - DNA, 20 bp, synthetic construct
  misc_feature 1..20: note = gRNA target site
  source 1..20: mol_type = other DNA
attgaaggagaagaccaagt

(19) United States
(12) Patent Application Publication
(10) Pub. No.: US 2025/0137009 A1
(43) Pub. Date: May 1, 2025

(54) NOVEL TRANSCRIPTIONAL REGULATOR 30S RIBOSOMAL PROTEIN REGULATES PHYSIOLOGICAL RESPONSES IN PLANT CELLS

(71) Applicant: UT-Battelle, LLC, Oak Ridge, TN (US)

(72) Inventors: Jin-Gui Chen, Oak Ridge, TN (US); Amith R. Devireddy, Orangeburg, SC (US); Wellington Muchero; Gerald A. Tuskan, Oak Ridge, TN (US)

(21) Appl. No.: 18/899,066

(22) Filed: Sep. 27, 2024

Related U.S. Application Data
(60) Provisional application No. 63/586,150, filed on Sep. 28, 2023.

(51) Int. Cl. C12N 15/82; C12N 9/22; C12N 15/11

(52) U.S. Cl. CPC C12N 15/8273; C12N 9/22; C12N 15/11; C12N 15/8205; C12N 15/8213; C12N 2310/20

(57) ABSTRACT

The present disclosure provides genetically modified plants, plant cells, or plant tissues wherein the plants, plant cells, or plant tissues comprise an exogenous nucleic acid comprising a nucleotide sequence encoding a 30S ribosomal protein or a homolog thereof, wherein the 30S ribosomal protein or the homolog thereof is expressed in the plant, plant cell, or plant tissue. The current disclosure provides methods of improving drought tolerance and water loss in a plant, plant cell, or plant tissue, the methods comprising an exogenous nucleic acid sequence encoding a 30S ribosomal protein or a homolog thereof, wherein the 30S ribosomal protein or the homolog thereof is expressed in the plant, plant cell, or plant tissue. Additionally, the current disclosure is describes to genetically modified plants, plant cells, or plant tissues wherein the plants, plant cells, or plant tissues comprise an exogenous nucleic acid comprising a nucleotide sequence encoding a cation/H+ exchanger 20 (CHX20) protein or a homolog thereof, wherein the nucleotide sequence is operably linked to a heterologous promoter, and the CHX20 protein or homolog thereof is expressed in the plant, plant cell, or plant tissue. Finally, the current disclosure provides methods of improving drought tolerance and water loss in a plant, plant cell, or plant tissue, the methods comprising an exogenous nucleic acid sequence encoding a CHX20 or a homolog thereof, wherein the CHX20 or the homolog thereof is expressed in the plant, plant cell, or plant tissue.

### CROSS REFERENCE TO RELATED APPLICATIONS

This application claims the benefit of U.S. Provisional Patent Application No. 63/586,150, filed Sep. 28, 2023, the contents of which is incorporated herein by reference in its entirety.

### STATEMENT REGARDING FEDERALLY SPONSORED RESEARCH OR DEVELOPMENT

This disclosure was made with government support under a research project supported by Prime Contract No. DE-AC05-00OR22725 awarded by the U.S. Department of Energy. The government has certain rights in this invention.

### INCORPORATION BY REFERENCE OF SEQUENCE LISTING

The Sequence Listing in an XML format, named as 42458_5200_1_SequenceListing.xml of 17,154 bytes, created on Sep. 23, 2024, and submitted to Patent Center, is incorporated herein by reference.

### BACKGROUND

Plants being sessile organisms are exposed to severe environmental conditions. Of all abiotic stresses that negatively influence plant life, drought/water deficit conditions are considered one of the major stress inducers and a critical environmental condition that is significantly reducing global plant productivity. Given the increasing impact of global climate change, water resources and availability will be critical factors in plant productivity worldwide. Drought conditions not only hinder plant growth, and development but also alter photosynthetic, and metabolic processes in plants. Some of these alterations include escalating leaf senescence, chlorosis, degradation of photosynthetic pigments resulting in reduced crop productivity. One of the efficient strategies that plant employ to tolerate drought is by reducing water loss and balancing photosynthetic carbon fixation by controlling their stomatal aperture. In this way plants regulate rates of transpiration and photosynthesis in parallel, maintaining a balance between gas exchange and assimilation rate thus helping them reduce yield penalties and maintain biomass accumulation during water deficit conditions.

Most plants on Earth facilitate gas exchange by opening and closing the stomatal pores. Stomatal pores are microscopic epidermal openings on the leaves and stems that are bounded by a pair of specialized epidermal cells known as guard cells. The guard cells control the size of the stomatal aperture, determining the extent and efficiency of the plant's photosynthetic carbon fixation. During water deficit or limited conditions stomatal closure is the first physiological response in most plants to limit moisture loss from leaves and water balance of the plants. Nevertheless, stomatal conductance directly modifies plant water relations and photosynthesis, particularly during severe drought episodes. Hence, there is an immediate need to enhance a plant's water-holding capacity plausibly by preventing water loss.

### SUMMARY OF THE DISCLOSURE

The current disclosure is directed to genetically modified plants, plant cells, or plant tissues wherein the plants, plant cells, or plant tissues comprise an exogenous nucleic acid comprising a nucleotide sequence encoding a 30S ribosomal protein or a homolog thereof, wherein the 30S ribosomal protein or the homolog thereof is expressed in the plant, plant cell, or plant tissue. The current disclosure is also directed to methods of improving drought tolerance and water loss in a plant, plant cell, or plant tissue, the methods comprising an exogenous nucleic acid sequence encoding a 30S ribosomal protein or a homolog thereof, wherein the 30S ribosomal protein or the homolog thereof is expressed in the plant, plant cell, or plant tissue. Additionally, the current disclosure is directed to genetically modified plants, plant cells, or plant tissues wherein the plants, plant cells, or plant tissues comprise an exogenous nucleic acid comprising a nucleotide sequence encoding a cation/H+ exchanger 20 (CHX20) protein or a homolog thereof, wherein the nucleotide sequence is operably linked to a heterologous promoter, and the CHX20 protein or homolog thereof is expressed in the plant, plant cell, or plant tissue. Finally, the current disclosure is directed to methods of improving drought tolerance and water loss in a plant, plant cell, or plant tissue, the methods comprising an exogenous nucleic acid sequence encoding a CHX20 or a homolog thereof, wherein the CHX20 or the homolog thereof is expressed in the plant, plant cell, or plant tissue.

One aspect of the present disclosure is directed to a genetically modified plant, plant cell, or plant tissue, comprising an exogenous nucleic acid which comprises a nucleotide sequence encoding a 30S ribosomal protein or a homolog thereof, wherein the 30S ribosomal protein or the homolog thereof is expressed in the plant, plant cell, or plant tissue.

In some embodiments, the genetically modified plant, plant cell, or plant tissue comprises an increase in expression of endogenous cation/H+ exchanger 20 (CHX20) gene or a homolog thereof in the plant, plant cell, or plant tissue as compared to CHX20 gene expression in a wild-type plant, plant cell, or plant tissue

In some embodiments, the nucleotide sequence has at least 90% sequence identity to the nucleotide sequence shown in SEQ ID NO: 1. In some embodiments, the 30S ribosomal protein or the homolog thereof has at least 90% sequence identity to the amino acid sequence shown in SEQ ID NO: 2.

In some embodiments, the exogenous nucleic acid is stably integrated into the plant genome. In some embodiments, the nucleotide sequence is operably linked to a heterologous promoter.

In some embodiments, the heterologous promoter is a constitutive promoter, an inducible promoter, or a native promoter. In some embodiments, the heterologous promoter is a 35S promoter.

In some embodiments, the plant is a monocot or a dicot.

In some embodiments, the plant is selected from the group consisting of genera Acer, Afzelia, Allium, Arabidopsis, Agrostis, Avena, Betula, Brassica, Capsicum, Citrullus, Cucumis, Eucalyptus, Fagus, Festuca, Fraxinus, Fragaria, Glycine, Gossypium, Hordeum, Ipomoea, Jatropha, Juglans, Lemna, Lolium, Malus, Manihot, Medicago, Micropus, Milium, Miscanthus, Nicotiana, Oryza, Pennisetum, Phalaris, Phleum, Picea, Pinus, Poa, Populus, Prunus, Quercus, Rosa, Salix, Solanum, Sorghum, Spinacia, Tectona, Trifolium, Triticum, Panicum, Saccharum, Setaria, Zea, and Zoysia.

In some embodiments, the plant is Arabidopsis. In some embodiments, the plant is Populus.

In some embodiments, the plant displays one or more of the following characteristics:
retains water within its cell by altering its stomatal aperture;
has altered stomatal aperture length compared to a wild type plant;
has improved gaseous exchange, transpiration, and photosynthesis under severe water deficit conditions compared to a wild type plant;
has higher survival rate under water deficit conditions compared to a wild type plant; and
has delayed leaf senescence in drought conditions compared to a wild type plant.

In some embodiments, the drought condition is a cyclic drought condition or a short-term drought condition.

Certain aspects of the disclosure are directed to a method of improving drought tolerance and water loss in a plant, plant cell, or plant tissue comprising an exogenous nucleic acid sequence encoding a 30S ribosomal protein or a homolog thereof, wherein the 30S ribosomal protein or the homolog thereof is expressed in the plant, plant cell, or plant tissue.

In some embodiments, the genetically modified plant, plant cell, or plant tissue comprises an increase in expression of endogenous CHX20 gene or a homolog thereof in the plant, plant cell, or plant tissue as compared to CHX20 gene expression in a wild-type plant, plant cell, or plant tissue.

In some embodiments, the nucleotide sequence has at least 90% sequence identity to the nucleotide sequence shown in SEQ ID NO: 1. In some embodiments, the 30S ribosomal protein or the homolog thereof has at least 90% sequence identity to the amino acid sequence shown in SEQ ID NO: 2.

In some embodiments, the exogenous nucleic acid is stably integrated into the plant genome. In some embodiments, the nucleotide sequence is operably linked to a heterologous promoter.

In some embodiments, the heterologous promoter is a constitutive promoter, an inducible promoter, or a native promoter. In some embodiments, the heterologous promoter is a 35S promoter.

In some embodiments, the plant is a monocot or a dicot.

In some embodiments, the plant is selected from the group consisting of genera Acer, Afzelia, Allium, Arabidopsis, Agrostis, Avena, Betula, Brassica, Capsicum, Citrullus, Cucumis, Eucalyptus, Fagus, Festuca, Fraxinus, Fragaria, Glycine, Gossypium, Hordeum, Ipomoea, Jatropha, Juglans, Lemna, Lolium, Malus, Manihot, Medicago, Micropus, Milium, Miscanthus, Nicotiana, Oryza, Pennisetum, Phalaris, Phleum, Picea, Pinus, Poa, Populus, Prunus, Quercus, Rosa, Salix, Solanum, Sorghum, Spinacia, Tectona, Trifolium, Triticum, Panicum, Saccharum, Setaria, Zea, and Zoysia.

In some embodiments, the plant is Arabidopsis. In some embodiments, the plant is Populus.

In some embodiments, the plant displays one or more of the following characteristics:
retains water within its cell by altering its stomatal aperture;
has altered stomatal aperture length compared to a wild type plant;
has improved gaseous exchange, transpiration, and photosynthesis under severe water deficit conditions compared to a wild type plant;
has higher survival rate under water deficit conditions compared to a wild type plant; and
has delayed leaf senescence in drought conditions compared to a wild type plant.

In some embodiments, the drought condition is a cyclic drought condition or a short-term drought condition.

Another aspect of this disclosure is directed to a genetically modified plant, plant cell, or plant tissue, comprising an exogenous nucleic acid which comprises a nucleotide sequence encoding a cation/H+ exchanger 20 (CHX20) protein or a homolog thereof, wherein the nucleotide sequence is operably linked to a heterologous promoter and the CHX20 protein or homolog thereof is expressed in the plant, plant cell, or plant tissue.

In some embodiments, the nucleotide sequence has at least 90% sequence identity to the nucleotide sequence shown in SEQ ID NO: 3.

In some embodiments, the CHX20 or the homolog thereof has at least 90% sequence identity to the amino acid sequence shown in SEQ ID NO: 4.

Another aspect of this disclosure is directed to a method of improving drought tolerance and water loss in a plant, plant cell, or plant tissue comprising an exogenous nucleic acid sequence encoding CHX20 or a homolog thereof, wherein the CHX20 or the homolog thereof is expressed in the plant, plant cell, or plant tissue.

In some embodiments, the genetically modified plant, plant cell, or plant tissue comprises an increase in expression of endogenous CHX20 gene or a homolog thereof in the plant, plant cell, or plant tissue as compared to CHX20 gene expression in a wild-type plant, plant cell, or plant tissue

In some embodiments, the nucleotide sequence has at least 90% sequence identity to the nucleotide sequence shown in SEQ ID NO: 3. In some embodiments, the 30S ribosomal protein or the homolog thereof has at least 90% sequence identity to the amino acid sequence shown in SEQ ID NO: 4.

In some embodiments, the exogenous nucleic acid is stably integrated into the plant genome. In some embodiments, the nucleotide sequence is operably linked to a heterologous promoter.

In some embodiments, the heterologous promoter is a constitutive promoter, an inducible promoter, or a native promoter. In some embodiments, the heterologous promoter is a 35S promoter.

In some embodiments, the plant is a monocot or a dicot.

In some embodiments, the plant is selected from the group consisting of genera Acer, Afzelia, Allium, Arabidopsis, Agrostis, Avena, Betula, Brassica, Capsicum, Citrullus, Cucumis, Eucalyptus, Fagus, Festuca, Fraxinus, Fragaria, Glycine, Gossypium, Hordeum, Ipomoea, Jatropha, Juglans, Lemna, Lolium, Malus, Manihot, Medicago, Micropus, Milium, Miscanthus, Nicotiana, Oryza, Pennisetum, Phalaris, Phleum, Picea, Pinus, Poa, Populus, Prunus, Quercus, Rosa, Salix, Solanum, Sorghum, Spinacia, Tectona, Trifolium, Triticum, Panicum, Saccharum, Setaria, Zea, and Zoysia.

In some embodiments, the plant is Arabidopsis. In some embodiments, the plant is Populus.

In some embodiments, the plant displays one or more of the following characteristics:
retains water within its cell by altering its stomatal aperture;
has altered stomatal aperture length compared to a wild type plant;
has improved gaseous exchange, transpiration, and photosynthesis under severe water deficit conditions compared to a wild type plant;
has higher survival rate under water deficit conditions compared to a wild type plant; and
has delayed leaf senescence in drought conditions compared to a wild type plant.

In some embodiments, the drought condition is a cyclic drought condition or a short-term drought condition.

### BRIEF DESCRIPTION OF THE DRAWINGS

FIG. 1A-1E. Transcriptional regulator 30S ribosomal protein potentially regulates CHX20 in plants. (A, B) Physiological response of five 4-week-old candidate Arabidopsis transcriptional regulator knockout lines under control and water deficit conditions, respectively. (C) Recovery of Arabidopsis transcriptional regulator knockout lines to 7 days of rewatering. (D) Expression level of CHX20 in WT and Arabidopsis knockout lines of 30S ribosomal protein. (E) Expression level of the 30S ribosomal protein in wild-type (WT) and Arabidopsis transgenic lines overexpressing the 30S ribosomal protein.

FIG. 2A-2E. A cation/H+ exchanger CHX20 is required for stomatal responses during drought stress in Arabidopsis thaliana. (A) Representative images of CHX20 mutant stomatal apertures in response to 21 days of water deficit condition. (B) Physiological response of CHX20 KO plants to drought stress. (C) Stomatal aperture response of CHX20 mutant lines in response to drought stress. (D) Stomatal conductance and transpiration measurements of CHX20 transgenic lines in response to drought stress. (E) Water loss of detached leaves of CHX20 transgenic lines.

FIG. 3A-3F. Overexpression of cation/H+ exchanger CHX20 enhances drought tolerance in Populus species. (A) Representative images of Populus transgenics (KO and OE) in response to 2-week water deficit condition. (B-D) Gas exchange measurements ((B) transpiration, (C) assimilation, and (D) stomatal conductance) of Populus CHX20 transgenics in response to drought stress. (E) Leaf water potential measurements of Populus CHX20 transgenics in response to drought stress conditions. (F) Water loss percentage of Populus CHX20 transgenics in response to drought stress conditions.

FIG. 4A-4D. Overexpression of 30S Ribosomal protein enhances tolerance to water deficit stress in Arabidopsis thaliana plants. (A-D). Gas exchange measurements including stomatal conductance (gsw) and transpiration rate (E) of Arabidopsis 30S overexpressing transgenics under control (A, C) and water deficit conditions (B, D) respectively.

### DETAILED DESCRIPTION

Ion transporters are required for efficient function (opening or closer) of stomatal aperture in plants. Especially during drought episodes or water deficit conditions, the efficiency of gas exchange and photosynthesis in plants heavily relies on the control of the stomatal apertures. Using GWAS (genome-wide association studies), QTL (quantitative trait loci) mapping, and transcriptomic analysis, a transcriptional regulator 30S ribosomal protein has been identified that potentially regulates a cation/H+ antiporter 20 (CHX20) that belongs in the CPA superfamily which is highly associated with drought-induced leaf senescence under drought stress. Functional validation of this gene using CHX20 knockout and overexpressing mutant lines in the model plant Arabidopsis and a bioenergy crop Populus suggests that CHX20 is required for proper stomatal responses during drought stress and overexpression of this gene enhances drought tolerance. The CHX20 overexpression lines efficiently maintained gaseous exchange, and water balance, and exhibited no yield penalties under drought conditions. Taken together, this disclosure provides a method to genetically engineer plants by manipulating the expression of 30S ribosomal protein and CHX20 to better adapt and tolerate drought environments, as a consequence of global climate change.

### Definitions

Unless defined otherwise, all technical and scientific terms used herein have the same meaning as commonly understood by one of ordinary skill in the art to which this invention belongs.

As used herein, the term “about” refers to an approximately +/−10% variation from a given value.

The term “control plant,” as used herein, refers to a plant of the same species that does not comprise the modification or modifications described in this disclosure. In some embodiments, the control plant is of the same variety. In some embodiments, the control plant is of the same genetic background.

The term “GWAS (genome-wide association studies)” as used herein, refers to a method to identify genetic variations that are associated with specific traits, or other phenotypes in a population. Such genetic variations can occur due to single nucleotide polymorphisms (SNPs). GWAS an observational study of a genome-wide set of genetic variants in different individuals to see if any variant is associated with a trait.

The term “Quantitative trait loci (QTL) mapping” as used herein, refers to a genetic analysis that seeks to understand the genetic basis of gene expression variation among individuals within a population.

The term “leaf senescence” as used herein, refers to the final stage of leaf development and is critical for plants' fitness as nutrient relocation from leaves to reproducing seeds is achieved through this process. Leaf senescence occurs by age-dependent internal factors and is also influenced by a range of other internal and environmental factors, including such as phytochrome, darkness, drought, pathogen attack, and oxidative stress.

As used herein, the term “drought stress” or “drought” refers to a sub-optimal environmental condition associated with limited availability of water to a plant. Limited availability of water may occur when, for instance, rain is absent or lower and/or when the plants are watered less frequently than required. Limited water availability to a plant may also occur when for instance water is present in soil but cannot efficiently be extracted by the plant. For instance, when soils strongly bind water or when the water has a high salt content, it may be more difficult for a plant to extract the water from the soil. Hence, many factors can contribute to result in limited availability of water, i.e., drought, to a plant. The effect of subjecting plants to “drought” or “drought stress” may be that plants do not have optimal growth and/or development. Plants subjected to drought may have wilting signs. For example, plants may be subjected to a period of at least 15 days under specific controlled conditions wherein no water is provided, e.g., without rain fall and/or watering of the plants.

As used herein, the term “cyclic drought” refers to a recurring occurrence of drought conditions spanning about 7-8 days separated by normal conditions.

As used herein, “short-term drought” refers to a weather pattern that results in precipitation deficit lasting for between weeks and less than six months.

The term “exogenous,” as used herein, refers to a substance or molecule originating or produced outside of an organism. The term “exogenous gene” or “exogenous nucleic acid molecule,” as used herein, refers to a nucleic acid that codes for the expression of an RNA and/or protein that has been introduced (“transformed”) into a cell or a progenitor of the cell. An exogenous gene may be from a different species (and so a “heterologous” gene) or from the same species (and so a “homologous” gene), relative to the cell being transformed. A transformed cell may be referred to as a recombinant or genetically modified cell. An “endogenous” nucleic acid molecule, gene, or protein can represent the organism's own gene or protein as it is naturally produced by the organism.

The term “expression” refers to the process of converting genetic information of a polynucleotide into RNA through transcription, which is catalyzed by an enzyme, RNA polymerase and into protein, through translation of mRNA on ribosomes. Expression can be, for example, constitutive or regulated, such as, by an inducible promoter (e.g., lac operon, which can be triggered by Isopropyl β-D-1-thiogalactopyranoside (IPTG). Up-regulation or “overexpression” refers to regulation that increases the production of expression products (mRNA, polypeptide or both) relative to basal or native states, while inhibition or down-regulation refers to regulation that decreases production of expression products (mRNA, polypeptide or both) relative to basal or native states. Expression of a gene can be measured through a suitable assay, such as real-time quantitative reverse transcription polymerase chain reaction (qRT-PCR), Northern blot, transcriptome sequencing and Western blot.

As used herein, overexpression of the target gene or protein means that the target protein is expressed more in the modified plant, plant cell, and/or plant tissue as compared to basal or native states of target protein expression in non-modified wild type plant, plant cell, and/or plant tissue. In some embodiments, the overexpressed target protein has 5-40-fold target protein expression relative to a wild type plant. In some embodiments, an overexpressed target protein has at least a 30% increase in expression of the target protein as compared to a wild type plant, i.e., at least 1.3× or 1.3-fold target protein expression relative to a wild type plant. In some embodiments, an overexpressed target protein has at least a 40% increase in expression as compared to a wild type plant. In some embodiments, an overexpressed target protein has at least a 50% increase in expression as compared to a wild type plant. In some embodiments, an overexpressed target protein has at least a 60% increase in expression as compared to a wild type plant. In some embodiments, an overexpressed target protein has at least a 70% increase in expression as compared to a wild type plant. In some embodiments, an overexpressed target protein has at least a 80% increase in expression as compared to a wild type plant. In some embodiments, an overexpressed target protein has at least a 90% increase in expression as compared to a wild type plant. In some embodiments, an overexpressed target protein has at least a 100% increase (i.e. 2-fold) in expression as compared to a wild type plant. In some embodiments, an overexpressed target protein has at least a 125% increase in expression as compared to a wild type plant. In some embodiments, an overexpressed target protein has at least a 150% increase in expression as compared to a wild type plant. In some embodiments, an overexpressed target protein has at least a 175% increase in expression as compared to a wild type plant. In some embodiments, an overexpressed target protein has at least a 200% increase in expression as compared to a wild type plant. In some embodiments, an overexpressed target protein has at least a 225% increase in expression as compared to a wild type plant. In some embodiments, an overexpressed target protein has at least a 250% increase in expression as compared to a wild type plant. In some embodiments, an overexpressed target protein has at least a 275% increase in expression as compared to a wild type plant. In some embodiments, an overexpressed target protein has at least a 300% increase in expression as compared to a wild type plant. In some embodiments, an overexpressed target protein is at least 5-fold relative to the amount of target protein in a wild type plant. In some embodiments, an overexpressed target protein is at least 10-fold relative to the amount of target protein in a wild type plant. In some embodiments, an overexpressed target protein is at least 15-fold relative to the amount of target protein in a wild type plant. In some embodiments, an overexpressed target protein is at least 20-fold relative to the amount of target protein in a wild type plant. In some embodiments, an overexpressed target protein is at least 25-fold relative to the amount of target protein in a wild type plant. In some embodiments, an overexpressed target protein is at least 30-fold relative to the amount of target protein in a wild type plant. In some embodiments, an overexpressed target protein is at least 35-fold relative to the amount of target protein in a wild type plant. In some embodiments, an overexpressed target protein is at least 40-fold relative to the amount of target protein in a wild type plant.

The term “gene,” as used herein, refers to a segment of nucleic acid that encodes an individual protein or RNA and can include both exons and introns together with associated regulatory regions such as promoters, operators, terminators, 5′ untranslated regions, 3′ untranslated regions, and the like.

The term “genetically modified” (or “genetically engineered” or “transgenic” or “cisgenic”) refers to a plant comprising a manipulated genome or nucleic acids. In some embodiments, the manipulation is the addition of exogenous nucleic acids to the plant. In some embodiments, the manipulation is changing the endogenous genes of the plant.

The term “homologous” refers to nucleic acids or polypeptides that are highly related at the level of nucleotide or amino acid sequence. Nucleic acids or polypeptides that are homologous to each other are termed “homologues.” The term “homolog” refers to a gene related to a second gene by descent from a common ancestral DNA sequence, therefore, the corresponding polynucleotide/polypeptide has a certain degree of homology, i.e., sequence identity (at least 40%, at least 60%, 65%, 66%, 68%, 70%, 75%, 80%, 86%, 88%, 90%, 92%, 95%, 97% or 99%). A “homolog” furthermore means that the function is equivalent to the function of the original gene. Homologs of a given gene and corresponding or equivalent positions in the homologous genes or proteins can be determined by sequence alignment programs, e.g., including but not limited to, NCBI BLAST, ClustalW, DIAMOND, CS-BLAST, and MAFFT.

The term “improved drought resistance” (or “drought tolerance”) refers to plants which, when provided with improved drought resistance, when subjected to drought or drought stress do not show effects or show alleviated effects as observed in control plants not provided with improved drought resistance. A normal plant has some level of drought resistance. It can easily be determined whether a plant has improved drought resistance by comparing a control plant with a plant provided with improved drought resistance under controlled conditions chosen such that in the control plants signs of drought can be observed after a certain period, i.e., when the plants are subjected to drought or drought stress. The plants with improved drought resistance will show less and/or reduced signs of having been subjected to drought, such as wilting, as compared to the control plants. The skilled person knows how to select suitable conditions. When a plant has “improved drought resistance,” it is capable of sustaining normal growth and/or normal development when being subjected to drought or drought stress would otherwise have resulted in reduced growth and/or reduced development of normal plants. Hence, “improved drought resistance” is determined by comparing plants, whereby the plant most capable of sustaining (normal) growth under drought stress is a plant with “improved drought resistance.” The skilled person is able to select appropriate conditions to determine drought resistance of a plant and how to measure signs of droughts, such as described in for example manuals by the IRRI, Breeding rice for drought prone environments, Fischer et al., 2003; and by the CIMMYT, Breeding for drought and nitrogen stress tolerance in maize: from theory to practice, Banzinger et al, 2000. Examples of methods for determining improved drought resistance in plants are provided in Snow and Tingey (1985, Plant Physiol, 77, 602-7) and Harb et al. (Analysis of drought stress in Arabidopsis, A O P 2010, Plant Physiology Review). In some embodiments, improvement is quantitatively measured. Several physiological parameters are known and used in the art as quantitative indicators of plant health during abiotic stresses. These parameters include relative water content (RWC), alternated rate of stomatal aperture, leaf surface temperature, and maximum quantum yield of photosystem II measured as Fᵥ/Fₘ. In some embodiments, improvement is measured as compared to a wild-type or “normal” plant, i.e. a plant which has not adopted the exogenous nucleic acid.

As used herein, the term “nucleic acid” has its general meaning in the art and refers to a coding or non-coding nucleic sequence. Nucleic acids include DNA (deoxyribonucleic acid) and RNA (ribonucleic acid) nucleic acids. Examples of nucleic acids thus include but are not limited to DNA, mRNA, tRNA, IRNA, tmRNA, miRNA, piRNA, snoRNA, and snRNA. Nucleic acids thus encompass coding and non-coding region of a genome (i.e., nuclear or mitochondrial or chloroplast).

The term “operably linked” refers to positioning of a regulatory region and a sequence to be transcribed in a nucleic acid so as to influence transcription or translation of such a sequence. For example, to bring a coding sequence under the control of a regulatory region, the translation initiation site of the translational reading frame of the polypeptide is typically positioned between one and about fifty nucleotides downstream of the promoter. A regulatory region can, however, be positioned as much as about 5,000 nucleotides upstream of the translation initiation site or about 2,000 nucleotides upstream of the transcription start site. A regulatory region typically comprises at least a core (basal) promoter.

The term “regulatory region” refers to a nucleic acid having nucleotide sequences that influence transcription or translation initiation and rate and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns and combinations thereof.

A regulatory region also may include at least one control element, such as an enhancer sequence, an upstream element or an upstream activation region (UAR). For example, a suitable enhancer is a cis-regulatory element (−212 to −154) from the upstream region of the octopine synthase (ocs) gene (Fromm et al., The Plant Cell, 1:977-984 (1989)). The choice of regulatory regions to be included depends upon several factors, including, but not limited to, efficiency, selectability, inducibility, desired expression level and cell- or tissue-preferential expression. It is a routine matter for one of skill in the art to modulate the expression of a coding sequence by appropriately selecting and positioning regulatory regions relative to the coding sequence.

As used herein, “vector” refers to a nucleic acid molecule into which a foreign nucleic acid molecule can be introduced without disrupting the ability of the vector to replicate and/or integrate in a host cell. A “vector” is a replicon, such as a plasmid, phage or cosmid, into which another DNA segment may be inserted so as to bring about the replication of the inserted segment. Generally, a vector is capable of replication when associated with the proper control elements. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. Suitable vector backbones include, for example, those routinely used in the art such as plasmids, viruses, artificial chromosomes, BACs, YACs or PACs. The term “vector” includes cloning and expression vectors, as well as viral vectors and integrating vectors.

A vector can also include one or more selectable marker genes and other genetic elements known in the art. An integrating vector is capable of integrating itself into a host nucleic acid. An “expression vector” is a vector that includes the necessary regulatory sequences to allow transcription and translation of inserted gene or genes. Suitable expression vectors include, without limitation, plasmids and viral vectors derived from, for example, bacteriophage, baculoviruses and retroviruses. Numerous vectors and expression systems are commercially available from such corporations as Novagen (Madison, Wis.), Clontech (Mountain View, Calif.), Stratagene (La Jolla, Calif.) and Invitrogen/Life Technologies (Carlsbad, Calif.).

One type of vector is a “plasmid,” which refers to a circular double stranded DNA loop into which additional DNA segments can be inserted, such as by standard molecular cloning techniques. Another type of vector is a viral vector, wherein virally derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a tobacco mosaic virus (TMV), potato virus X (PVX), tobacco rattle virus (TRV), barley stripe mosaic virus (BSMV) or geminivirus vector. In some embodiments the geminiviral vector is a bean yellow dwarf virus vector or tomato yellow leaf curl virus.

Methods of transforming plants are known in the art. Transformation of a plant includes increasing or decreasing expression of a target gene and/or polypeptide. In some embodiments, the transformation is a stable transformation. As used herein, “stable transformation” means that the gene will be fully integrated into the host genome and is expressed continuously. The gene in a stable transformation will also be expressed in later generations, or progeny, of the plant. Progeny includes descendants of a particular plant or plant line provided the progeny inherits the transgene. Progeny of a plant include seeds formed on F1, F2, F3, F4, F5, F6 and subsequent generation plants or seeds formed on BC1, BC2, BC3 and subsequent generation plants or seeds formed on F1BC1, F1BC2, F1BC3 and subsequent generation plants. There are numerous proven genetic transformation methods in the art that can stably introduce new genes into the nuclear genomes of different plant species. Exogenous genes can be delivered to plant cells by Agrobacterium, particle bombardment/gene gun, electroporation, the pollen tube pathway, and other known mediated delivery methods.

### Plants

There is no specific limitation on the plants that can be used in the methods of the present disclosure, as long as the plant is suitable to be transformed by a gene. The term “plant,” as used herein, includes whole plants, plant tissues or plant cells. The plants that can be used for the methods and compositions of the present disclosure include various crops, flower plants or plants of forestry, etc. Specifically, the plants include, but are not limited to, dicotyledon, monocotyledon or gymnosperm. More specifically, the plants include, but is not limited to, wheat, barley, rye, rice, corn, sorghum, beet, apple, pear, plum, peach, apricot, cherry, strawberry, Rubus swinhoei Hance, blackberry, bean, lentil, pea, soy, rape, mustard, opium poppy, olea europea, helianthus, coconut, plant producing castor oil, cacao, peanut, calabash, cucumber, watermelon, cotton, flax, cannabis, jute, citrus, lemon, grapefruit, spinach, lettuce, asparagus, cabbage, Brassica campestris L. ssp. Pekinensis, Brassica campestris L. ssp. chinensis, carrot, onion, murphy, tomato, green pepper, avocado, cassia, camphor, tobacco, nut, coffee, eggplant, sugar cane, tea, pepper, grapevine, nettle grass, banana, natural rubber tree and ornamental plant, etc.

In some embodiment the methods and compositions of the present disclosure are also be used over a broad range of plant species from the dicot genera Acer, Afzelia, Arabidopsis, Betula, Brassica, Eucalyptus, Fagus, Fraxinus, Glycine, Gossypium, Jatropha, Juglans, Linum, Lycopersicon, Medicago, Micropus, Populus, Prunus, Quercus, Salix, Solanum, Tectona and Trifolium; and the monocot genera Agrostis, Avena, Festuca, Hordeum, Lemna, Lolium, Milium, Miscanthus, Oryza, Panicum, Pennisetum, Phalaris, Phleum, Poa, Saccharum, Secale, Sorghum, Triticum, Zea and Zoysia; and the gymnosperm genera Abies, Picea and Pinus. In some embodiments, a plant is a member of the species Festuca arundinacea, Miscanthus hybrid (Miscanthus×giganteus), Miscanthus sinensis, Miscanthus sacchariflorus, Panicum virgatum, Pennisetum purpureum, Phalaris arundinacea, Populus spp including but not limited to balsamifera, deltoides, tremuloides, tremula, alba and maximowiczii, Saccharum spp., Secale cereale, Sorghum almum, Sorghum halcapense or Sorghum vulgare. In certain embodiments, the polynucleotides and vectors described herein can be used to transform a number of monocotyledonous and dicotyledonous plants and plant cell systems, wherein such plants are hybrids of different species.

In some embodiments, the plant for the methods and compositions of the present disclosure is a C3 plant. The term “C3 plant” refers to a plant that captures carbon dioxide into three-carbon compounds to enter into the Calvin cycle (photosynthesis pathway). In a C3 plant carbon dioxide capture and the Calvin cycle occur during the daytime, and stomata of C3 plants are open during the day for gas exchange, which also leads to increased water loss through the stomata (evapotranspiration). In some embodiment, the C3 plant is selected from the group consisting of genera Allium, Arabidopsis, Brassica, Capsicum, Citrullus, Cucumis, Eucalyptus, Fragaria, Glycine, Gossypium, Hordeum, Ipomoea, Malus, Manihot, Nicotiana, Oryza, Populus, Prunus, Rosa, Solanum, Spinacia and Triticum.

In some embodiments, the plant for the methods and compositions of the present disclosure is a C4 plant. The term “C4 plant” refers to a plant that captures carbon dioxide into four-carbon compounds to enter into the Calvin cycle. In a C4 plant carbon dioxide capture and the Calvin cycle occur during the daytime, and stomata of C4 plants are open during the day for gas exchange, which also leads to increased water loss. In some embodiment, the C4 plant is selected from the group consisting of genera Panicum, Saccharum, Setaria, Sorghum and Zea.

### Expression Vectors

The polynucleotides and expression vectors described herein can be used to increase the expression of a 30S ribosomal protein and the cation/H+ exchanger CHX20 in plants and render them tolerant under drought/water deficit conditions.

The vectors provided herein can include origins of replication, scaffold attachment regions (SARs) and/or markers. A marker gene can confer a selectable phenotype on a plant cell. For example, a marker can confer biocide resistance, such as resistance to an antibiotic (e.g., kanamycin, G418, bleomycin or hygromycin) or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin or Flag-tag (Kodak, New Haven, Conn.) sequences typically are expressed as a fusion with the encoded polypeptide. Such tags can be inserted anywhere within the polypeptide, including at either the carboxyl or amino terminus. As described herein, plant cells can be transformed with a recombinant nucleic acid construct to express a polypeptide of interest.

### Promoters

A variety of promoters are available for use, depending on the degree of expression desired. For example, a broadly expressing promoter promotes transcription in many, but not necessarily all, plant tissues. Non-limiting examples of broadly expressing promoters that can be included in the nucleic acid constructs provided herein include the cauliflower mosaic virus (CaMV) 35S promoter, the mannopine synthase (MAS) promoter, the l′ or 2′ promoters derived from T-DNA of Agrobacterium tumefaciens, the figwort mosaic virus 34S promoter, actin promoters such as the rice actin promoter and ubiquitin promoters such as the maize ubiquitin-1 promoter.

In some embodiments, the promoter to drive expression of genes of interest is a constitutive promoter. In some embodiments the constitutive promoter is selected from the group consisting of a ubiquitin promoter, a cauliflower mosaic virus (CaMV) 35S promoter, an actin promoter, a peanut chlorotic streak caulimovirus promoter, a Chlorella virus methyltransferase gene promoter, a full-length transcript promoter form figwort mosaic virus, a pEMU promoter, a MAS promoter, a maize H3 histone promoter and an Agrobacterium gene promoter.

In some embodiments, the promoter to drive expression of genes of interest is a regulated promoter. In some embodiments the regulated promoter is selected from the group consisting of a stress induced promoter, chemical-induced promoter, a light induced promoter, a dark-induced promoter, and a circadian-clock controlled promoter.

Some suitable regulatory regions initiate transcription, only or predominantly, in certain cell types. For instance, promoters active in photosynthetic tissue confer transcription in green tissues such as leaves and stems. Examples of such promoters include the ribulose-1,5-bisphosphate carboxylase (RbcS) promoters such as the RbcS promoter from eastern larch (Larix laricina), the pine chlorophyll a/b binding-6 (cab6) promoter (Yamamoto et al., 1994, Plant Cell Physiol., 35:773-778), the chlorophyll a/b binding-1 (Cab-1) promoter from wheat (Fejes et al., 1990, Plant Mol. Biol., 15:921-932), the chlorophyll a/b binding-1 (CAB-1) promoter from spinach (Lubberstedt et al., 1994, Plant Physiol., 104:997-1006), the cab IR promoter from rice (Luan et al., 1992, Plant Cell, 4:971-981), the pyruvate orthophosphate dikinase (PPDK) promoter from corn (Matsuoka et al., 1993. Proc. Natl. Acad. Sci. USA, 90:9586-9590), the tobacco light-harvesting complex of photosystem (Lhcb1*2) promoter (Cerdan et al., 1997, Plant Mol. Biol., 33:245-), the Arabidopsis SUC2 sucrose-H+ symporter promoter (Truernit et al., 1995, Planta, 196:564-570) and thylakoid membrane protein promoters from spinach (psaD, psaF, psaE, PC, FNR, atpC, atpD, cab, rbcS).

In some embodiments, promoters of the instant application comprise inducible promoters. Inducible promoters confer transcription in response to external stimuli such as chemical agents or environmental stimuli. For example, inducible promoters can confer transcription in response to hormones such as gibberellic acid or ethylene or in response to light, nitrogen, shade or drought.

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

A 5′ untranslated region (UTR) can be included in nucleic acid constructs described herein. A 5′ UTR is transcribed but is not translated and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a vector, e.g., introns, enhancers, upstream activation regions, transcription terminators and inducible elements. Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

Techniques for introducing nucleic acids into monocotyledonous and dicotyledonous plants are known in the art and include, without limitation, Agrobacterium-mediated transformation, viral vector-mediated transformation, electroporation and particle gun transformation, e.g., U.S. Pat. Nos. 5,538,880, 5,204,253, 6,329,571 and 6,013,863, incorporated herein by reference in their entirety. If a cell or tissue culture is used as the recipient tissue for transformation, plants can be regenerated from transformed cultures if desired, by techniques known to those skilled in the art. See, e.g., Niu et al., 2000. Plant Cell Rep. V19: 304-310; Chang and Yang, 1996, Bot. Bull. Acad. Sin., V37: 35-40; and Han et al., 1999, Biotechnology in Agriculture and Forestry, V44: 291 (ed. by Y. P. S. Bajaj), Springer-Vernag.

### The Transcriptional Regulatory Role of 30S Ribosomal Protein to Tolerate Drought/Water Deficit Conditions in Plants

Disclosed herein are plants and plant cells genetically modified by introduction of the disclosed exogenous nucleic acids and expression vectors to display increased tolerance under drought/water deficit condition.

Typically, genetically modified plant cells used in methods described herein constitute part or all of a whole plant. Such plants can be grown in a manner suitable for the species under consideration, either in a growth chamber, a greenhouse or in a field. Genetically modified plants can be bred as desired for a particular purpose, e.g., to introduce a recombinant nucleic acid into other lines, to transfer a recombinant nucleic acid to other species or for further selection of other desirable traits. Progeny includes descendants of a particular plant or plant line provided the progeny inherits the transgene. Progeny of a plant include seeds formed on F1, F2, F3, F4, F5, F6 and subsequent generation plants or seeds formed on BC1, BC2, BC3 and subsequent generation plants or seeds formed on F1BC1, F1BC2, F1BC3 and subsequent generation plants. Seeds produced by a genetically modified plant can be grown and then selfed (or outcrossed and selfed) to obtain seeds homozygous for the nucleic acid construct. Alternatively, genetically modified plants can be propagated vegetatively for those species amenable to such techniques.

Based on the segregating drought-induced phenotype at the Boardman field site in Oregon, a few poplar trees were identified that were able to maintain green leaf tissue under drought stress conditions. These trees were then scored based on the severity of leaf senescence. Genome-wide association studies (GWAS), and quantitative trait loci (QTL) mapping, revealed candidate genes of which a plasma membrane localized 30S ribosomal protein known to be involved in signal transduction process in the model plant Arabidopsis thaliana. Further analysis using GWAS, QTL mapping, and transcriptomic analysis indicated that the 30S ribosomal protein could regulate a cis-regulatory element highly associated with delayed leaf senescence under drought stress. These studies suggested that drought-induced leaf senescence is regulated by a single locus cation/H+ exchanger 20 (CHX20) in the Populus GWAS population. CHX20 belongs in the CPA superfamily thought to be involved in the osmoregulation through K (+) fluxes and pH modulation of an active endomembrane system in guard cells. To further understand and functionally validate the role of this 30S ribosomal protein and ion transporter in mediating drought stress responses, Arabidopsis knockout lines of five candidate genes including 30S ribosomal protein sourced from the Arabidopsis Biological Resource Center (ABRC) were studied. Additionally, knockout and overexpression lines of CHX20 in Arabidopsis and Populus species were generated.

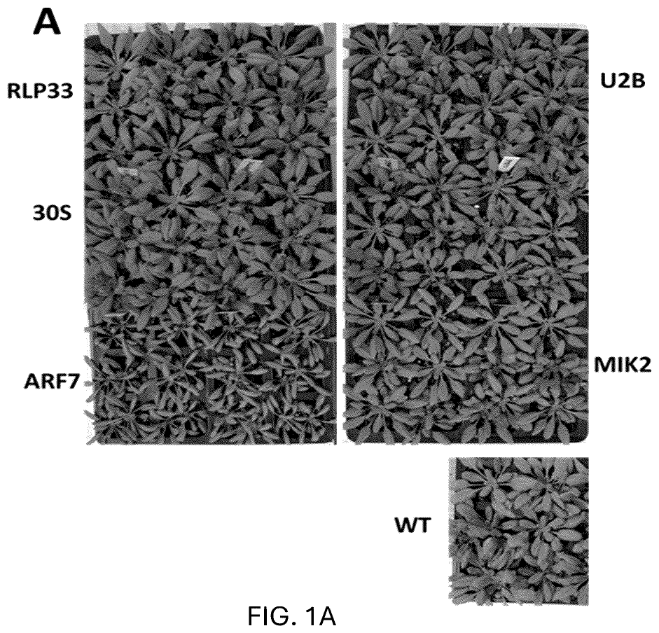

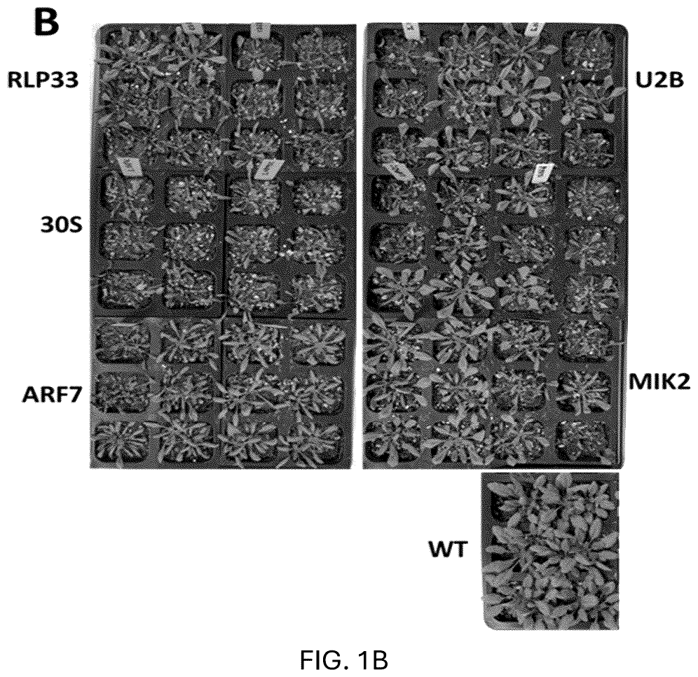

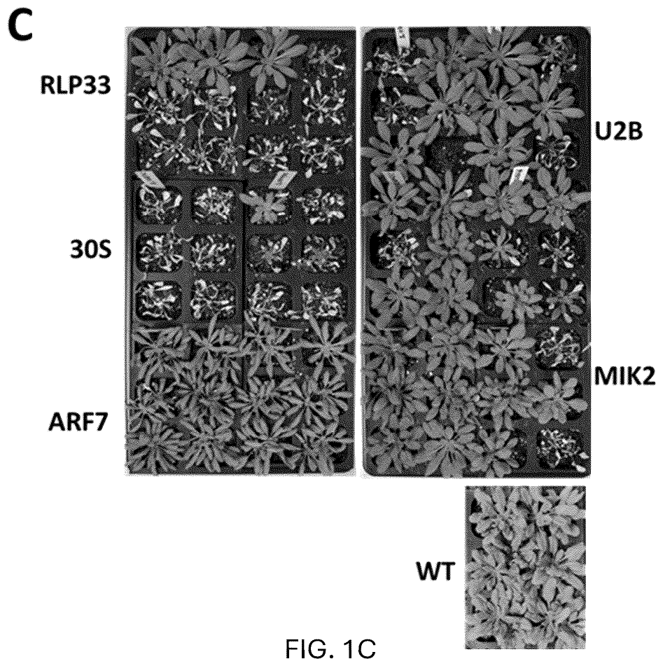

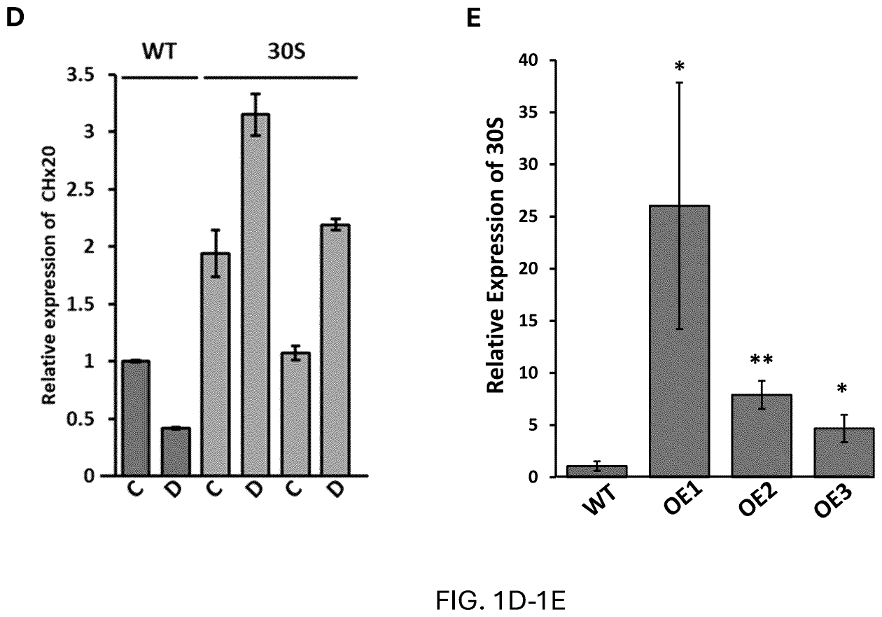

The knockout lines of 30S ribosomal protein showed no morphological difference under control/well-watered conditions (FIG. 1A), however under water deficit conditions, these knockout lines showed enhanced water loss and resulted in higher cell death compared to wild type (FIG. 1B). Similarly, when these lines were rewatered for 7 days and checked for recovery/survival, the KO lines of 30S ribosomal protein had low survival rate compared to wild type (FIG. 1C). In line with these observations, the expression level of CHX20 was altered in the 30S ribosomal protein knockout lines when plants were subjected to drought treatment suggesting that this protein could potentially regulate CHX20 in plants (FIG. 1D).

In some embodiments, the genetically modified plant, plant cell, or plant tissue, comprises an exogenous nucleotide sequence encoding 30S ribosomal protein or a homolog thereof, wherein the 30S ribosomal protein or the homolog thereof is expressed in the plant, plant cell, or plant tissue. In some embodiments, the genetically modified plant, plant cell or plant comprises an increase in expression of endogenous cation/H+ exchanger 20 (CHX20) gene or a homolog thereof in the plant, plant cell, or plant tissue as compared to CHX20 gene expression in a wild-type plant, plant cell, or plant tissue.

In some embodiments, the nucleotide sequence is laid out as in SEQ ID NO: 1. In some embodiments, the nucleotide sequence has at least 90% sequence identity to the nucleotide sequence shown in SEQ ID NO: 1. In some embodiments, the nucleotide sequence has at least 91% sequence identity to the nucleotide sequence shown in SEQ ID NO: 1. In some embodiments, the nucleotide sequence has at least 92% sequence identity to the nucleotide sequence shown in SEQ ID NO: 1. In some embodiments, the nucleotide sequence has at least 93% sequence identity to the nucleotide sequence shown in SEQ ID NO: 1. In some embodiments, the nucleotide sequence has at least 94% sequence identity to the nucleotide sequence shown in SEQ ID NO: 1. In some embodiments, the nucleotide sequence has at least 95% sequence identity to the nucleotide sequence shown in SEQ ID NO: 1. In some embodiments, the nucleotide sequence has at least 96% sequence identity to the nucleotide sequence shown in SEQ ID NO: 1. In some embodiments, the nucleotide sequence has at least 97% sequence identity to the nucleotide sequence shown in SEQ ID NO: 1. In some embodiments, the nucleotide sequence has at least 98% sequence identity to the nucleotide sequence shown in SEQ ID NO: 1. In some embodiments, the nucleotide sequence has at least 99% sequence identity to the nucleotide sequence shown in SEQ ID NO: 1.

In some embodiments, the 30S ribosomal protein or homolog thereof has an amino acid sequence as laid out in SEQ ID NO: 2. In some embodiments, the 30S ribosomal protein or the homolog thereof has at least 90% sequence identity to the amino acid sequence shown in SEQ ID NO: 2. In some embodiments, the 30S ribosomal protein or the homolog thereof has at least 91% sequence identity to the amino acid sequence shown in SEQ ID NO: 2. In some embodiments, the 30S ribosomal protein or the homolog thereof has at least 92% sequence identity to the amino acid sequence shown in SEQ ID NO: 2. In some embodiments, the 30S ribosomal protein or the homolog thereof has at least 93% sequence identity to the amino acid sequence shown in SEQ ID NO: 2. In some embodiments, the 30S ribosomal protein or the homolog thereof has at least 94% sequence identity to the amino acid sequence shown in SEQ ID NO: 2. In some embodiments, the 30S ribosomal protein or the homolog thereof has at least 95% sequence identity to the amino acid sequence shown in SEQ ID NO: 2. In some embodiments, the 30S ribosomal protein or the homolog thereof has at least 96% sequence identity to the amino acid sequence shown in SEQ ID NO: 2. In some embodiments, the 30S ribosomal or the homolog thereof has at least 97% sequence identity to the amino acid sequence shown in SEQ ID NO: 2. In some embodiments, the 30S ribosomal protein or the homolog thereof has at least 98% sequence identity to the amino acid sequence shown in SEQ ID NO: 2. In some embodiments, the 30S ribosomal protein or the homolog thereof has at least 99% sequence identity to the amino acid sequence shown in SEQ ID NO: 2.

In some embodiments, the exogenous nucleic acid is stably integrated into the plant genome. Stably transformed cells typically retain the introduced nucleic acid with each cell division. The stably transformed genetically modified plants, plant cells or plant tissue can be useful in the methods described herein.

In some embodiments, the nucleotide sequence is operably linked to a heterologous promoter. The heterologous promoter can be a constitutive promoter, an inducible promoter, or a native promoter. In some embodiments, the heterologous promoter is a 35S promoter.

In some embodiments, the plant is a monocot or a dicot. In some embodiments, genetically modified plant, plant cell, or plant tissue according to any one of claims 1-9, wherein the plant is selected from the group consisting of genera Acer, Afzelia, Allium, Arabidopsis, Agrostis, Avena, Betula, Brassica, Capsicum, Citrullus, Cucumis, Eucalyptus, Fagus, Festuca, Fraxinus, Fragaria, Glycine, Gossypium, Hordeum, Ipomoea, Jatropha, Juglans, Lemna, Lolium, Malus, Manihot, Medicago, Micropus, Milium, Miscanthus, Nicotiana, Oryza, Pennisetum, Phalaris, Phleum, Picea, Pinus, Poa, Populus, Prunus, Quercus, Rosa, Salix, Solanum, Sorghum, Spinacia, Tectona, Trifolium, Triticum, Panicum, Saccharum, Setaria, Zea, and Zoysia. In some embodiments, the plant is Arabidopsis. In some embodiments, the plant is Populus.

In some embodiments, the genetically modified plant, plant cell, or plant tissue displays one or more of the following characteristics as compared to a non-modified/wild type plant, plant cell, or plant tissue: retains water within its cell by altering its stomatal aperture; has altered stomatal aperture length compared to a wild type plant; has improved gaseous exchange, transpiration, and photosynthesis under severe water deficit conditions compared to a wild type plant; has higher survival rate under water deficit conditions compared to a wild type plant; and has delayed leaf senescence in drought conditions compared to a wild type plant. In some embodiments, the drought condition is a cyclic drought condition or a short-term drought condition spanning about 7-8 days. In the case of cyclic drought condition, the recurring appearance of drought conditions are separated by normal conditions.

In some embodiments, the method of improving drought tolerance and water loss in a plant, plant cell, or plant tissue comprises an exogenous nucleic acid sequence encoding a 30S ribosomal protein or a homolog thereof, wherein the 30S ribosomal protein or the homolog thereof is expressed in the plant, plant cell, or plant tissue.

In some embodiments, the extent of improvement of drought tolerance is measured phenotypically. In some embodiments, the extent of improvement of drought tolerance is determined by comparing plants, whereby the plant most capable of sustaining (normal) growth under drought stress is a plant with “improved drought resistance.” The skilled person is able to select appropriate conditions to determine drought resistance of a plant and how to measure signs of drought, such as described in, for example, manuals by the IRRI, Breeding rice for drought prone environments, Fischer et al., 2003; and by the CIMMYT, Breeding for drought and nitrogen stress tolerance in maize: from theory to practice, Banzinger et al, 2000. Examples of methods for determining improved drought resistance in plants are also provided in Snow and Tingey (1985, Plant Physiol, 77, 602-7) and Harb et al. (Analysis of drought stress in Arabidopsis, A O P 2010, Plant Physiology Review). In some embodiments, the use of the known methods for assessing drought tolerance is quantified. Several physiological parameters are known and used in the art as quantitative indicators of plant health during abiotic stresses. These parameters include relative water content (RWC), stomatal opening rate/stomatal conductance, transpiration rate, assimilation rate, leaf water potential, water loss percentage, leaf surface temperature, and maximum quantum yield of photosystem II measured as Fᵥ/Fₘ. In some embodiments, improvement is measured as compared to a wild-type or “normal” plant, i.e. a plant which has not adopted the exogenous nucleic acid.

In some embodiments, the improvement in transpiration rate in a plant overexpressed with 30S is at least about 1.5-25× (i.e., 1.5 to 25-fold) as compared to a wild type plant, i.e., at least 50% increase relative to a wild type plant. In some embodiments, the improvement in transpiration rate in a plant overexpressed with 30S is at least about 2× as compared to a wild type plant. In some embodiments, the improvement in transpiration rate in a plant overexpressed with 30S is at least about 2.25× as compared to a wild type plant. In some embodiments, the improvement in transpiration rate in a plant overexpressed with 30S is at least about 2.5× as compared to a wild type plant. In some embodiments, the improvement in transpiration rate in a plant overexpressed with 30S is at least about 2.75× as compared to a wild type plant. In some embodiments, the improvement in transpiration rate in a plant overexpressed with 30S is at least about 3× as compared to a wild type plant. In some embodiments, the improvement in transpiration rate in a plant overexpressed with 30S is at least about 3.25× as compared to a wild type plant. In some embodiments, the improvement in transpiration rate in a plant overexpressed with 30S is at least about 3.5× as compared to a wild type plant. In some embodiments, the improvement in transpiration rate in a plant overexpressed with 30S is at least about 3.75× as compared to a wild type plant. In some embodiments, the improvement in transpiration rate in a plant overexpressed with 30S is at least about 4× as compared to a wild type plant. In some embodiments, the improvement in transpiration rate in a plant overexpressed with 30S is at least about 4.25× as compared to a wild type plant. In some embodiments, the improvement in transpiration rate in a plant overexpressed with 30S is at least about 4.5× as compared to a wild type plant. In some embodiments, the improvement in transpiration rate in a plant overexpressed with 30S is at least about 4.75× as compared to a wild type plant. In some embodiments, the improvement in transpiration rate in a plant overexpressed with 30S is at least about 5× as compared to a wild type plant.

In some embodiments, the improvement in assimilation rate in a plant overexpressed with 30S is at least about 2× (i.e. 2-fold) as compared to a wild type plant. In some embodiments, the improvement in assimilation rate in a plant overexpressed with 30S is at least about 2.5× as compared to a wild type plant. In some embodiments, the improvement in assimilation rate in a plant overexpressed with 30S is at least about 3× as compared to a wild type plant. In some embodiments, the improvement in assimilation rate in a plant overexpressed with 30S is at least about 3.5× as compared to a wild type plant. In some embodiments, the improvement in assimilation rate in a plant overexpressed with 30S is at least about 4× as compared to a wild type plant. In some embodiments, the improvement in assimilation rate in a plant overexpressed with 30S is at least about 4.5× as compared to a wild type plant. In some embodiments, the improvement in assimilation rate in a plant overexpressed with 30S is at least about 5× as compared to a wild type plant. In some embodiments, the improvement in assimilation rate in a plant overexpressed with 30S is at least about 5.5× as compared to a wild type plant. In some embodiments, the improvement in assimilation rate in a plant overexpressed with 30S is at least about 6× as compared to a wild type plant. In some embodiments, the improvement in assimilation rate in a plant overexpressed with 30S is at least about 6.5× as compared to a wild type plant. In some embodiments, the improvement in assimilation rate in a plant overexpressed with 30S is at least about 7× as compared to a wild type plant.

In some embodiments, improvement in stomatal conductance in a plant overexpressed with 30S is at least about 2× (i.e. 2-fold) as compared to a wild type plant. In some embodiments, improvement in stomatal conductance in a plant overexpressed with 30S is at least about 2.5× as compared to a wild type plant. In some embodiments, improvement in stomatal conductance in a plant overexpressed with 30S is at least about 3× as compared to a wild type plant. In some embodiments, improvement in stomatal conductance in a plant overexpressed with 30S is at least about 3.5× as compared to a wild type plant. In some embodiments, improvement in stomatal conductance in a plant overexpressed with 30S is at least about 4× as compared to a wild type plant. In some embodiments, improvement in stomatal conductance in a plant overexpressed with 30S is at least about 4.5× as compared to a wild type plant. In some embodiments, improvement in stomatal conductance in a plant overexpressed with 30S is at least about 5× as compared to a wild type plant. In some embodiments, improvement in stomatal conductance in a plant overexpressed with 30S is at least about 5.5× as compared to a wild type plant. In some embodiments, improvement in stomatal conductance in a plant overexpressed with 30S is at least about 6× as compared to a wild type plant. In some embodiments, improvement in stomatal conductance in a plant overexpressed with 30S is at least about 6.5× as compared to a wild type plant. In some embodiments, improvement in stomatal conductance in a plant overexpressed with 30S is at least about 7× as compared to a wild type plant. In some embodiments, improvement in stomatal conductance in a plant overexpressed with 30S is at least about 7.5× as compared to a wild type plant. In some embodiments, improvement in stomatal conductance in a plant overexpressed with 30S is at least about 8× as compared to a wild type plant. In some embodiments, improvement in stomatal conductance in a plant overexpressed with 30S is at least about 8.5× as compared to a wild type plant. In some embodiments, improvement in stomatal conductance in a plant overexpressed with 30S is at least about 9× as compared to a wild type plant. In some embodiments, improvement in stomatal conductance in a plant overexpressed with 30S is at least about 9.5× as compared to a wild type plant. In some embodiments, improvement in stomatal conductance in a plant overexpressed with 30S is at least about 10× as compared to a wild type plant. In some embodiments, improvement in stomatal conductance in a plant overexpressed with 30S is at least about 11× as compared to a wild type plant. In some embodiments, improvement in stomatal conductance in a plant overexpressed with 30S is at least about 12× as compared to a wild type plant. In some embodiments, improvement in stomatal conductance in a plant overexpressed with 30S is at least about 13× as compared to a wild type plant. In some embodiments, improvement in stomatal conductance in a plant overexpressed with 30S is at least about 14× as compared to a wild type plant.

In some embodiments, the difference in water retention, measured as water loss, in a plant overexpressed with CHX20 is at least about 3 percentage points as compared to a wild type plant. In some embodiments, the difference in water retention in a plant overexpressed with CHX20 is at least about 3.5 percentage points as compared to a wild type plant. In some embodiments, the difference in water retention in a plant overexpressed with CHX20 is at least about 4 percentage points as compared to a wild type plant. In some embodiments, the difference in water retention in a plant overexpressed with CHX20 is at least about 4.5 percentage points as compared to a wild type plant. In some embodiments, the difference in water retention in a plant overexpressed with CHX20 is at least about 5 percentage points as compared to a wild type plant. In some embodiments, the difference in water retention in a plant overexpressed with CHX20 is at least about 5.5 percentage points as compared to a wild type plant. In some embodiments, the difference in water retention in a plant overexpressed with CHX20 is at least about 6 percentage points as compared to a wild type plant.

### The Putative Role of the Cation/H+ Exchanger CHX20 to Tolerate Drought/Water Deficit Conditions in Arabidopsis Plants

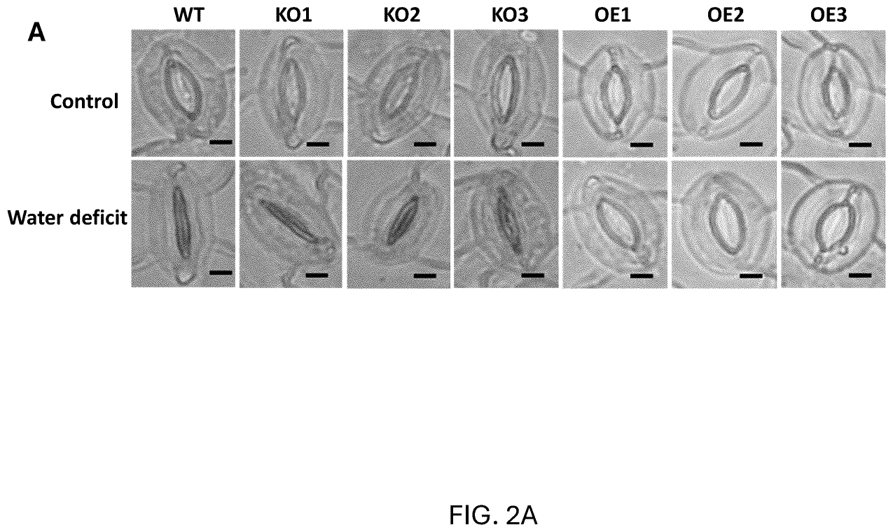

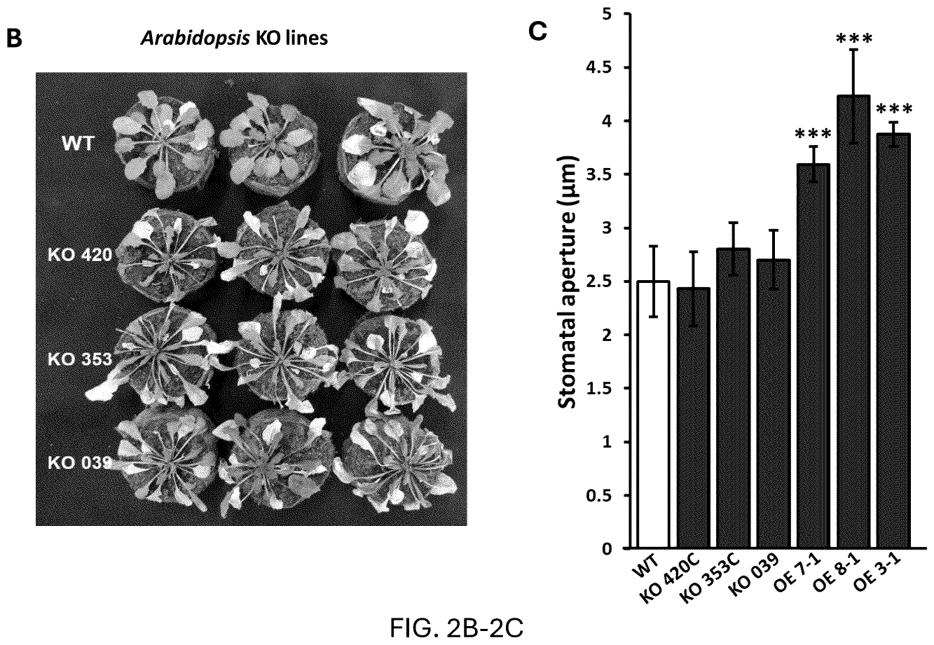

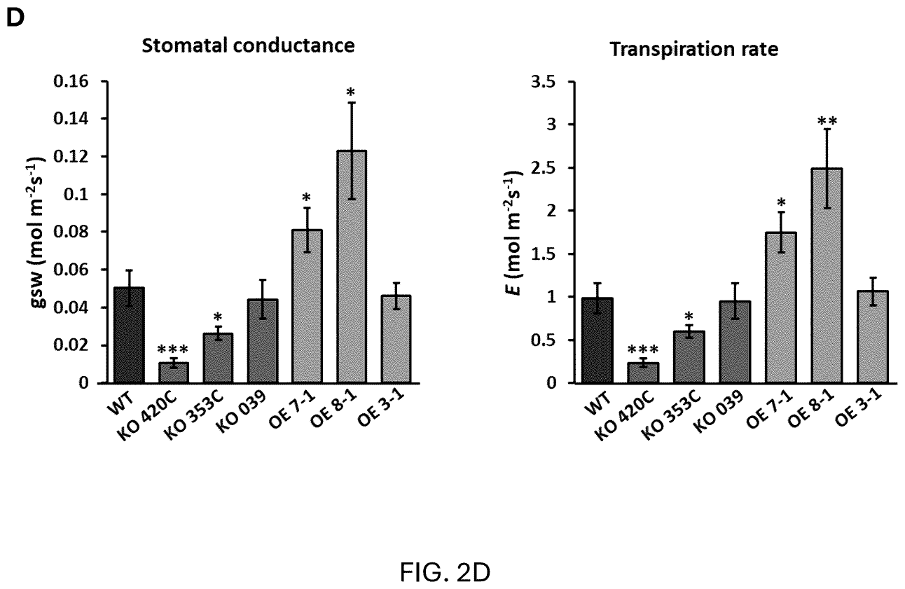

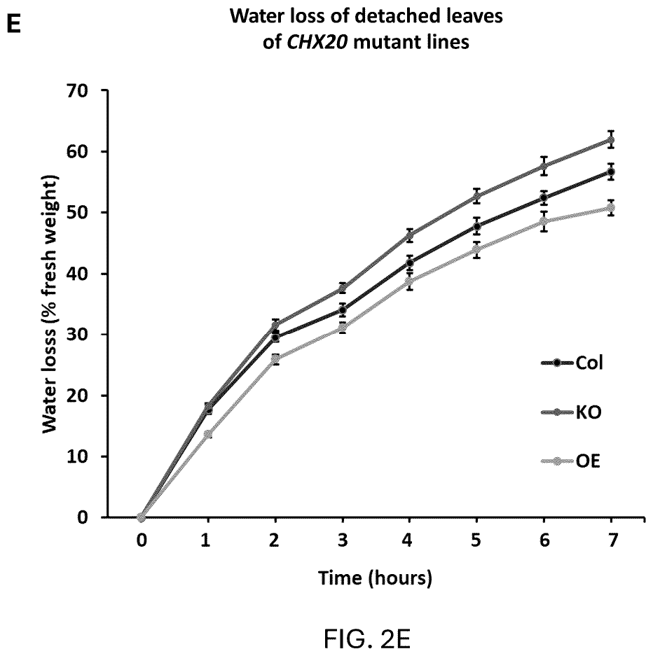

Under normal or unstressed conditions, the stomatal aperture lengths of CHX20 overexpression lines (OE) are significantly higher than the wild-type and CHX20 knockout mutants (FIGS. 2A and 2C). This would enable OE plants to perform enhanced gas exchange processes compared to the WT or KO plants. In contrast under severe drought stress conditions where plants were at 40% field capacity by withholding water for 21 days, the stomatal aperture of OE lines tended to be significantly higher than WT and KO lines (FIG. 2C). This would enable the OE plants to efficiently perform physiological functions such as gaseous exchange, transpiration, and photosynthesis under severe water deficit conditions (FIG. 2D).

Maintaining a proper water balance is required for plants for their development and growth. Water loss using detached leaves of CHX20 mutants suggested that OE plants tended to lose significantly less water compared to WT or KO plants. This signified that the OE lines could retain more water within their cell as compared to a wild type plant due to the OE lines altering their stomatal aperture (FIGS. 2B and 2E).

Biomass is an important trait to preserve during water deficit conditions. The water retention capacity of OE lines enabled plants to hold more water (indicated as fresh and dry weights) than WT or KO lines in both controlled and drought conditions.

### The Putative Role of the Cation/H⁺ Exchanger CHX20 to Tolerate Drought/Water Deficit Conditions in Populus Plants

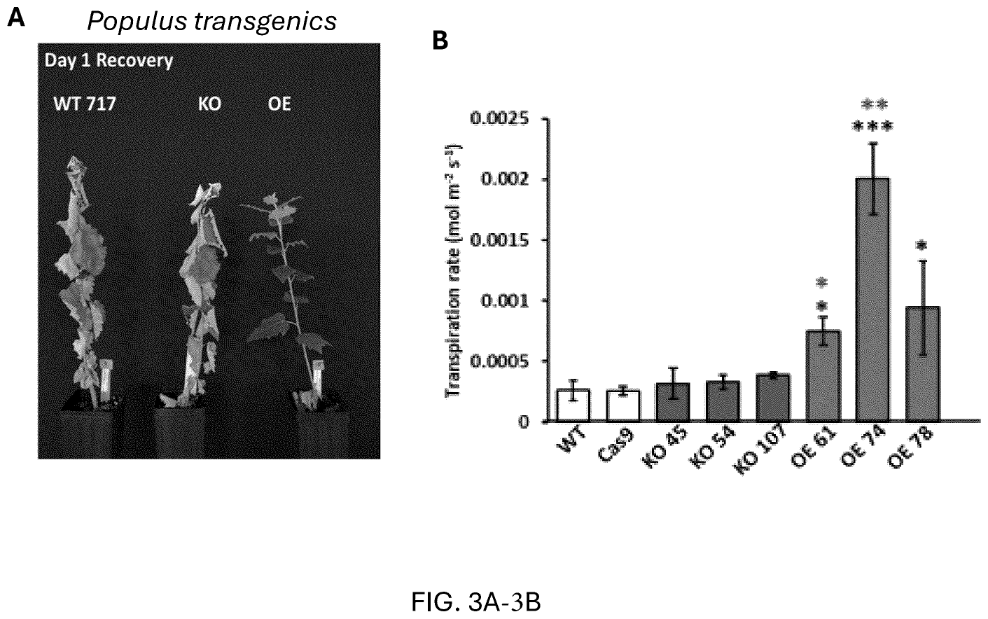

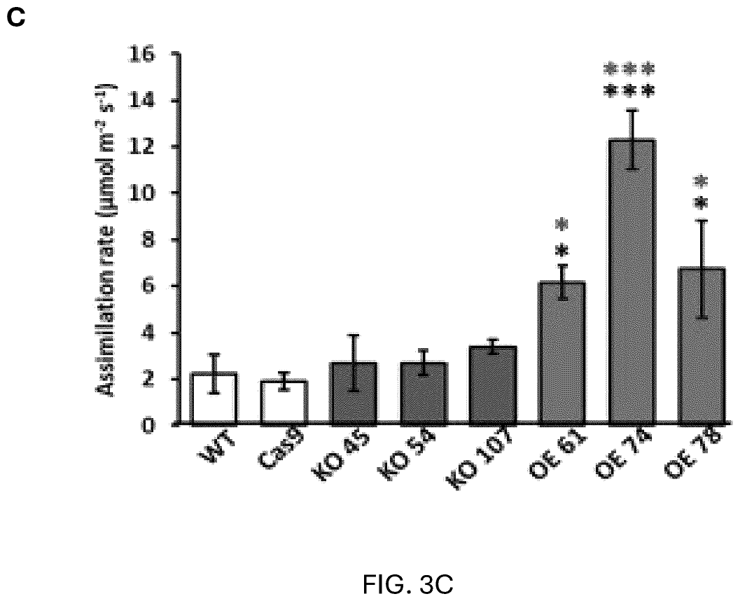

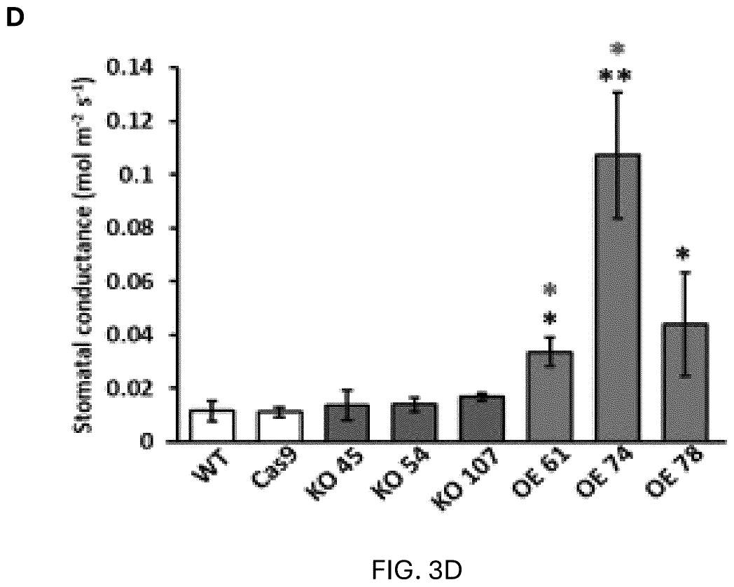

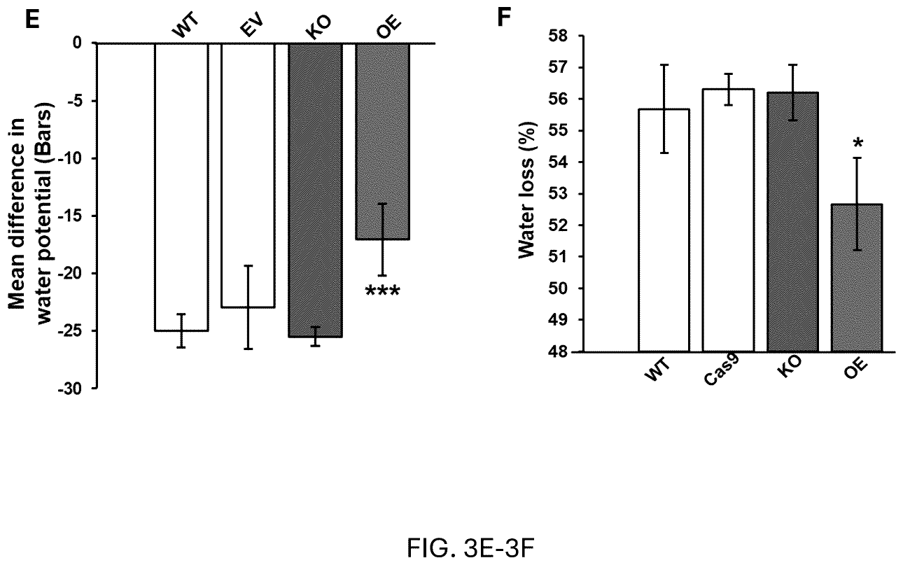

Under severe drought stress conditions where water was withheld for 10 or 14 days, the photosynthetic parameters such as gaseous exchange, transpiration, assimilation, stomatal conductance, photosynthesis, and leaf temperature (measured using Licor 6800) of Poplar CHX20 overexpression lines (OE) were significantly higher than the wild type and CHX20 knockout mutants (FIG. 3B-3D). This would enable transgenic CHX20 OE plants to withstand severe drought conditions and better aid in their survival.

Maintaining a proper water balance is required for plants for their development and growth. Under drought stress conditions, leaf water potential measured using the scholander pressure chamber and pot weight measurements suggested that CHX20 OE plants hold higher water potential (FIG. 3E) and water content (FIG. 3F) compared to WT, empty vector control, and KO plants. This indicated that CHX20 OE plants tend to lose significantly less water compared to WT or KO plants plausibly by shutting down the transpiration rate during drought conditions (FIG. 3A). Similarly, the survival and recovery rate of Poplar CHX20 OE lines were significantly greater than WT and/or KO lines under drought stress conditions.

In some embodiments, the genetically modified plant, plant cell, or plant tissue, comprises an exogenous nucleotide sequence encoding a cation/H+ exchanger 20 (CHX20) protein or a homolog thereof, wherein the nucleotide sequence is operably linked to a heterologous promoter and the CHX20 protein or homolog thereof is expressed in the plant, plant cell, or plant tissue.

In some embodiments, the nucleotide sequence is laid out in SEQ ID NO: 3. In some embodiments, the nucleotide sequence has at least 90% sequence identity to the nucleotide sequence shown in SEQ ID NO: 3. In some embodiments, the nucleotide sequence has at least 90% sequence identity to the nucleotide sequence shown in SEQ ID NO: 3. In some embodiments, the nucleotide sequence has at least 91% sequence identity to the nucleotide sequence shown in SEQ ID NO: 3. In some embodiments, the nucleotide sequence has at least 92% sequence identity to the nucleotide sequence shown in SEQ ID NO: 3. In some embodiments, the nucleotide sequence has at least 93% sequence identity to the nucleotide sequence shown in SEQ ID NO: 3. In some embodiments, the nucleotide sequence has at least 94% sequence identity to the nucleotide sequence shown in SEQ ID NO: 3. In some embodiments, the nucleotide sequence has at least 95% sequence identity to the nucleotide sequence shown in SEQ ID NO: 3. In some embodiments, the nucleotide sequence has at least 96% sequence identity to the nucleotide sequence shown in SEQ ID NO: 3. In some embodiments, the nucleotide sequence has at least 97% sequence identity to the nucleotide sequence shown in SEQ ID NO: 3. In some embodiments, the nucleotide sequence has at least 98% sequence identity to the nucleotide sequence shown in SEQ ID NO: 3. In some embodiments, the nucleotide sequence has at least 99% sequence identity to the nucleotide sequence shown in SEQ ID NO: 3.

In some embodiments, the CHX20 or the homolog thereof has an amino acid as laid out in SEQ ID NO: 4. In some embodiments, the CHX20 or the homolog thereof has at least 90% sequence identity to the amino acid sequence shown in SEQ ID NO: 4. In some embodiments, the CHX20 or the homolog thereof has at least 90% sequence identity to the amino acid sequence shown in SEQ ID NO: 4. In some embodiments, the CHX20 or the homolog thereof has at least 91% sequence identity to the amino acid sequence shown in SEQ ID NO: 4. In some embodiments, the CHX20 or the homolog thereof has at least 92% sequence identity to the amino acid sequence shown in SEQ ID NO: 4. In some embodiments, the CHX20 or the homolog thereof has at least 93% sequence identity to the amino acid sequence shown in SEQ ID NO: 4. In some embodiments, the CHX20 or the homolog thereof has at least 94% sequence identity to the amino acid sequence shown in SEQ ID NO: 4. In some embodiments, the CHX20 or the homolog thereof has at least 95% sequence identity to the amino acid sequence shown in SEQ ID NO: 4. In some embodiments, the CHX20 or the homolog thereof has at least 96% sequence identity to the amino acid sequence shown in SEQ ID NO: 4. In some embodiments, the CHX20 or the homolog thereof has at least 97% sequence identity to the amino acid sequence shown in SEQ ID NO: 4. In some embodiments, the CHX20 or the homolog thereof has at least 98% sequence identity to the amino acid sequence shown in SEQ ID NO: 4. In some embodiments, the CHX20 or the homolog thereof has at least 99% sequence identity to the amino acid sequence shown in SEQ ID NO: 4.

In some embodiments, the method of improving drought tolerance and water loss in a plant, plant cell, or plant tissue comprises an exogenous nucleic acid sequence encoding a cation/H+ exchanger 20 (CHX20) or a homolog thereof, wherein the CHX20 or the homolog thereof is expressed in the plant, plant cell, or plant tissue.

The 30S ribosomal protein potentially regulates a cation/H+ antiporter CHX20 that belongs in the CPA superfamily. The cation/H+ antiporter CHX20 in plants which would be used to enhance a plant's gas exchange parameters resulting in enhanced photosynthesis of economically important bioenergy crops such as Populus during severe drought stress conditions, and thus 30S ribosomal protein regulates physiological responses in plant cells. The disclosed methods allow for the following phenotypes in genetically modified Populus species as compared to wild type Populus species: (1) enhanced stress tolerance to drought conditions with limited or no yield penalties; (2) optimal gas exchange and photosynthesis by controlling the stomatal aperture under drought conditions; (3) enhanced plant water status; and (4) higher plant survival rate in drought conditions.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

### Example 1: Generation of Overexpression Lines of 30S Ribosomal Protein

The Coding Sequence (CDS) of At30S ribosomal protein was amplified using At30S-gib-F and At30S-gib-R to replace the Cas9 sequence in the p201N vector (Addgene, plasmid #59175). The amplified At30S and p201N backbone devoid of cas9 was reconstructed by Gibson assembly (E5510) to generate the 35S::At30S construct. Agrobacterium strain GV3101 transfected with the construct was used to transform Arabidopsis wild-type Col-0 plants using the floral dip method. The transformed seeds are selected by Kanamycin antibiotic resistance and regenerated to yield overexpression plants.

The primers used for Gibson cloning of 30S

At30S-gib-F:
(SEQ ID NO: 5)
TCTATCTCTCTCGACCGCTATGGCAACGCTTTTAGGTTT,
At30S-gib-R:
(SEQ ID NO: 6)
TCGAACCACTTTGTACAAGACTCGGCGAAAGAGTGTTCAT.
At30S sequence attached with GFP (green
fluorescence protein) sequence:
(SEQ ID NO: 7)
ATGGCAACGCTTTTAGGTTTCTCTCAGACTAAGTTTCACCATTGCGGCGT
CTGGATTTCTACGCCGCCGTGCTCATCTTCTTCCACGGTAGTCTCAATGG
TGGGCTTGAATCGGACTGATTCGAAAAAGCTCCGTTCAGATTTCCTCGGC
CAGATTGGTTACGAAGATCGGAGACAAGTAAGACACAGTTACTGTAGAAG
CTCATTAGCTGTGAAAATGTCGTGGGACGGTCCTCTTGCTTCCGTCAAGT
TGATCATCCAAGGAAAAAACCTCGAGTTATCAGAGCCAATTAAGCAGCAT
GTTGAAGAGAAAGTAGGCAAATCTGTTCAGAAACACAGTCATCTTGTGAG
AGAAGTTGATGTAAGACTCTCTGTTCGTGGTGGAGAGTTTGGTAAAGGCC
CTAGGATTCGAAGATGTGAGGTGACATTGTTTACAAAGAAGCATGGTGTT
GTGCGTGCTGAGGAAGATGCTGAGACAGTATACGCTTGTATCGACTTGGT
ATCAACGATAATACAGAGGAAGCTGAGGAAGATCAAGGAGAAGGACTCAG
ACCATGGAAGGCACATGAAAGGTTTCAACAGATTGAAGGTAAGGGAACCA
GTGATTGAGCCGGTTGTGGAGGATGTTGAGGACAGTACTGACTCGAGCGT
AGGAGAAGAAGAAGAAGAGGATGATTTGATCAAGGAGATTGTCCGTACCA
AGACTTTCGAGATGCCACCATTGACTGTCGCTGAGGCAGTCGAGCAGCTG
GAACTAGTCAGTCACGACTTCTATGGCTTCCAAAATGAAGAAACTGGTGA
GATAAACATAGTGTACAAGAGAAAAGAAGGAGGTTACGGTCTGATAATCC
CAAAGAAAGACGGGAAGGCCGAGAAGGTTGAGCCGCTTCCAACCGAGCAA
TTGAATGAACACTCTTTCGCCGAGTCTAGTACAAAGTGGTTCGATCTAGA
GGATCCATGGTGagcaagggcgaggagctgttcaccggggtggtgcccat
cctggtcgagctggacggcgacgtgaacggccacaagttcagcgtgtccg
gcgagggcgagggcgatgccacctacggcaagctgaccctgaagttcatc
tgcaccaccggcaagctgcccgtgccctggcccaccctcgtgaccacctt
cacctacggcgtgcagtgcttcagccgctaccccgaccacatgaagcagc
acgacttcttcaagtccgccatgcccgaaggctacgtccaggagcgcacc
atcttcttcaaggacgacggcaactacaagacccgcgccgaggtgaagtt
cgagggcgacaccctggtgaaccgcatcgagctgaagggcatcgacttca
aggaggacggcaacatcctggggcacaagctggagtacaactacaacagc
cacaacgtctatatcatggccgacaagcagaagaacggcatcaaggtgaa
cttcaagatccgccacaacatcgaggacggcagcgtgcagctcgccgacc
actaccagcagaacacccccatcggcgacggccccgtgctgctgcccgac
aaccactacctgagcacccagtccgccctgagcaaagaccccaacgagaa
gcgcgatcacatggtcctgctggagttcgtgaccgccgccgggatcactc
acggcatggacgagctgtacaagtaa

Capital letters represent At30S sequence (except the underlined region which are linkers added to separate the gene from GFP without having a stop codon.)
Small letters represent eGFP sequence.

The GFP tagging would help to better understand and visually track the expression of the gene in living plant cells and tissues and to understand the subcellular localization of the protein within plant cells.

SEQ ID NO: 1
Full length CDS of a 30S ribosomal protein

| | |
|---|---|
| 1 | ATGGCAACGC TTTTAGGTTT CTCTCAGACT AAGTTTCACC |
| | ATTGCGGCGT |
| 51 | CTGGATTTCT ACGCCGCCGT GCTCATCTTC TTCCACGGTA |
| | GTCTCAATGG |
| 101 | TGGGCTTGAA TCGGACTGAT TCGAAAAAGC TCCGTTCAGA |
| | TTTCCTCGGC |
| 151 | CAGATTGGTT ACGAAGATCG GAGACAAGTA AGACACAGTT |
| | ACTGTAGAAG |
| 201 | CTCATTAGCT GTGAAAATGT CGTGGGACGG TCCTCTTGCT |
| | TCCGTCAAGT |
| 251 | TGATCATCCA AGGAAAAAAC CTCGAGTTAT CAGAGCCAAT |
| | TAAGCAGCAT |
| 301 | GTTGAAGAGA AAGTAGGCAA ATCTGTTCAG AAACACAGTC |
| | ATCTTGTGAG |
| 351 | AGAAGTTGAT GTAAGACTCT CTGTTCGTGG TGGAGAGTTT |
| | GGTAAAGGCC |
| 401 | CTAGGATTCG AAGATGTGAG GTGACATTGT TTACAAAGAA |
| | GCATGGTGTT |
| 451 | GTGCGTGCTG AGGAAGATGC TGAGACAGTA TACGCTTGTA |
| | TCGACTTGGT |
| 501 | ATCAACGATA ATACAGAGGA AGCTGAGGAA GATCAAGGAG |
| | AAGGACTCAG |
| 551 | ACCATGGAAG GCACATGAAA GGTTTCAACA GATTGAAGGT |
| | AAGGGAACCA |
| 601 | GTGATTGAGC CGGTTGTGGA GGATGTTGAG GACAGTACTG |
| | ACTCGAGCGT |
| 651 | AGGAGAAGAA GAAGAAGAGG ATGATTTGAT CAAGGAGATT |
| | GTCCGTACCA |
| 701 | AGACTTTCGA GATGCCACCA TTGACTGTCG CTGAGGCAGT |
| | CGAGCAGCTG |
| 751 | GAACTAGTCA GTCACGACTT CTATGGCTTC CAAAATGAAG |
| | AAACTGGTGA |
| 801 | GATAAACATA GTGTACAAGA GAAAAGAAGG AGGTTACGGT |
| | CTGATAATCC |
| 851 | CAAAGAAAGA CGGGAAGGCC GAGAAGGTTG AGCCGCTTCC |
| | AACCGAGCAA |
| 901 | TTGAATGAAC ACTCTTTCGC CGAGTAG |

SEQ ID NO: 2
Amino acid sequence of a 30S ribosomal protein

| | |
|---|---|
| 1 | MATLLGFSQT KFHHCGVWIS TPPCSSSSTV VSMVGLNRTD |
| | SKKLRSDFLG |
| 51 | QIGYEDRRQV RHSYCRSSLA VKMSWDGPLA SVKLIIQGKN |
| | LELSEPIKQH |
| 101 | VEEKVGKSVQ KHSHLVREVD VRLSVRGGEF GKGPRIRRCE |
| | VTLFTKKHGV |
| 151 | VRAEEDAETV YACIDLVSTI IQRKLRKIKE KDSDHGRHMK |
| | GFNRLKVREP |
| 201 | VIEPVVEDVE DSTDSSVGEE EEEDDLIKEI VRTKTFEMPP |
| | LTVAEAVEQL |
| 251 | ELVSHDFYGF QNEETGEINI VYKRKEGGYG LIIPKKDGKA |
| | EKVEPLPTEQ |
| 301 | LNEHSFAE |

### Example 2: Generation of Arabidopsis and Populus CHX20 Overexpression Transgenic Lines

The CDS of AtCHX20 was amplified to replace Cas9 in the p201N vector by Gibson assembly to generate the 35S: AtCHX20 construct. Agrobacterium strain GV3101 transfected with the construct was used to transform Arabidopsis wild-type Col-0 plants using the floral dip method.

To generate Populus CHX20 transgenic KO lines, the design of a gene-specific and variant-free gRNA spacer for CRISPR-KO in Poplar 717 followed established practices. The gRNA target site (ATTGAAGGAGAAGACCAAGT (SEQ ID NO: 8)) was located within the second exon. A pair of oligos containing the spacer sequence and vector tails were assembled into p201N-Cas9 for Agrobacterium tumefaciens-mediated 717 transformation. A total of 30 independent primary transformants, along with WT and Cas9-only vector control (without gRNA), were subjected to amplicon sequencing and all 30 events were determined by AGEseq to harbor biallelic KO mutations. Five events with biallelic frameshift edits were propagated for experiments and three were subjected to physiological characterization in the greenhouse.

To generate PtCHX20 OE plants, the PtCHX20 coding sequence was PCR amplified from the CDS sequence of P. trichocarpa ‘Nisqually-1’ v3.0 from Phytozome and subcloned into p201N-Cas9 to replace Cas9. Transformation of 717 was done and leaves of tissue-cultured transformants were screened by qRT-PCR to assess transgene overexpression levels. Three lines with the highest OE levels were propagated for further experiments.

SEQ ID NO: 3
Full length CDS of a CHX20

| | |
|---|---|
| 1 | ATGCCCTTCA ACATAACCTC CGTGAAAACC TCATCTAACG |
| | GAGTATGGCA |
| 51 | AGGCGACAAT CCTTTAAACT TCGCTTTTCC GTTACTCATC |
| | GTCCAAACGG |
| 101 | CGTTAATCAT CGCCGTCAGT CGCTTCCTCG CCGTCTTATT |
| | CAAACCTCTC |
| 151 | CGTCAACCCA AAGTCATCGC CGAGATTGTC GGAGGGATTT |
| | TGTTAGGACC |
| 201 | ATCGGCTTTA GGTAGAAACA TGGCGTACAT GGACCGTATA |
| | TTTCCGAAAT |
| 251 | GGAGTATGCC GATACTCGAA TCCGTCGCGA GCATAGGACT |
| | TCTCTTCTTC |
| 301 | CTCTTCCTCG TCGGTCTAGA ACTCGATTTA TCATCGATCC |
| | GACGAAGCGG |
| 351 | CAAACGCGCT TTCGGAATCG CAGTCGCTGG AATTACACTA |
| | CCGTTTATCG |
| 401 | CCGGCGTCGG AGTCGCGTTT GTGATCCGTA ACACTCTCTA |
| | CACCGCCGCG |
| 451 | GATAAACCAG GTTACGCCGA GTTTCTCGTT TTCATGGGAG |
| | TCGCACTCTC |
| 501 | GATCACAGCT TTTCCGGTAC TTGCGCGTAT TTTAGCAGAG |
| | CTCAAGCTTT |
| 551 | TAACGACTCA GATAGGAGAA ACCGCGATGG CTGCAGCCGC |
| | TTTTAACGAT |
| 601 | GTAGCCGCGT GGATTTTACT CGCTTTAGCG GTTGCGTTAG |
| | CGGGTAATGG |
| 651 | CGGTGAGGGA GGTGGAGAGA AAAAGAGTCC GTTAGTGTCG |
| | TTGTGGGTTT |
| 701 | TGTTATCGGG CGCTGGGTTT GTGGTTTTTA TGTTGGTTGT |
| | GATCCGACCC |
| 751 | GGAATGAAAT GGGTCGCGAA ACGTGGATCT CCTGAAAACG |
| | ACGTCGTACG |
| 801 | CGAGTCTTAC GTGTGTTTGA CGTTAGCCGG TGTTATGGTT |
| | TCCGGTTTCG |
| 851 | CGACGGATTT AATTGGGATT CATTCGATTT TTGGAGCGTT |
| | TGTTTTCGGT |
| 901 | TTGACTATAC CGAAAGATGG AGAGTTTGGT CAGCGATTGA |
| | TTGAACGAAT |
| 951 | TGAGGATTTT GTTTCCGGTT TACTCTTACC GCTTTATTTC |
| | GCTACGAGTG |
| 1001 | GTTTGAAGAC TGACGTGGCT AAGATTAGAG GAGCTGAGTC |
| | GTGGGGGATG |
| 1051 | TTGGGTCTTG TTGTTGTTAC GGCTTGTGCC GGGAAGATAG |
| | TCGGAACTTT |
| 1101 | TGTTGTGGCG GTGATGGTTA AAGTTCCGGC GAGAGAGGCG |
| | TTGACACTTG |
| 1151 | GTTTCTTGAT GAATACTAAA GGTTTAGTGG AGCTCATTGT |
| | ACTCAACATA |
| 1201 | GGCAAGGAGA AAAAGGTACT AAACGACGAG ACGTTTGCAA |
| | TACTAGTGCT |
| 1251 | AATGGCACTC TTCACAACGT TCATAACGAC GCCTACTGTA |
| | ATGGCCATTT |
| 1301 | ACAAGCCGGC ACGTGGCACC CACCGCAAAC TAAAAGACTT |
| | GTCGGCGAGC |
| 1351 | CAAGACTCCA CCAAGGAAGA GCTTCGCATC CTAGCCTGCC |
| | TCCACGGCCC |
| 1401 | AGCCAATGTC TCCTCCCTCA TCTCTCTCGT CGAGTCCATC |
| | CGAACCACCA |
| 1451 | AGATACTACG GCTAAAGCTG TTTGTGATGC ATCTGATGGA |
| | ACTAACGGAA |
| 1501 | CGGTCTTCGT CAATCATAAT GGTGCAAAGA GCCCGTAAAA |
| | ACGGACTTCC |
| 1551 | TTTCGTTCAC CGTTACCGTC ATGGTGAGCG TCACAGCAAC |
| | GTCATAGGAG |
| 1601 | GCTTCGAAGC CTATCGTCAA CTAGGCCGGG TCGCAGTCCG |
| | GCCCATCACC |
| 1651 | GCAGTCTCTC CATTACCCAC AATGCACGAA GACATTTGCC |
| | ACATGGCAGA |
| 1701 | TACCAAGAGG GTCACAATGA TCATTTTACC TTTCCACAAA |
| | CGATGGAACG |
| 1751 | CTGATCATGG TCATAGCCAC CACCACCAAG ACGGAGGAGG |
| | AGATGGAAAC |
| 1801 | GTACCGGAAA ACGTTGGTCA TGGTTGGCGA TTGGTTAACC |
| | AAAGGGTTTT |
| 1851 | GAAGAATGCG CCGTGTTCGG TGGCGGTTCT TGTAGACCGT |
| | GGACTTGGGT |
| 1901 | CCATTGAGGC CCAAACTTTG AGCTTAGATG GGTCGAATGT |
| | GGTTGAAAGG |
| 1951 | GTTTGTGTGA TTTTCTTTGG TGGGCCTGAT GACCGTGAGT |
| | CTATAGAGCT |
| 2001 | CGGCGGGAGA ATGGCTGAGC ATCCGGCCGT TAAAGTTACC |
| | GTTATTAGGT |
| 2051 | TTTTGGTAAG AGAAACGTTG AGGAGTACCG CCGTCACTTT |
| | ACGACCGGCA |
| 2101 | CCGTCTAAAG GCAAGGAGAA GAACTATGCC TTTTTAACAA |
| | CCAACGTGGA |
| 2151 | TCCAGAAAAA GAAAAGGAAT TAGACGAAGG GGCATTGGAA |
| | GACTTCAAGA |
| 2201 | GCAAATGGAA AGAAATGGTG GAGTACAAAG AAAAGGAACC |
| | AAACAACATC |
| 2251 | ATTGAAGAAA TACTGTCAAT AGGACAGAGT AAAGACTTTG |
| | ATCTAATAGT |
| 2301 | GGTTGGAAGA GGGAGGATAC CGTCGGCCGA GGTGGCGGCA |
| | TTAGCTGAGC |
| 2351 | GTCAAGCTGA ACATCCTGAG TTAGGTCCTA TCGGAGACGT |
| | GCTCGCCTCT |
| 2401 | TCGATCAACC ACATCATTCC ATCAATCCTT GTGGTTCAAC |
| | AACACAACAA |
| 2451 | AGCTCATGTA GAGGATATTA CGGTTTCCAA AATTGTTAGT |
| | GAGTCTTCTC |
| 2501 | TAAGTATTAA CGGAGACACA AATGTATGA |

SEQ ID NO: 4
Amino acid sequence of a CHX20

| | |
|---|---|
| 1 | MPFNITSVKT SSNGVWQGDN PLNFAFPLLI VQTALIIAVS |
| | RFLAVLFKPL |
| 51 | RQPKVIAEIV GGILLGPSAL GRNMAYMDRI FPKWSMPILE |
| | SVASIGLLFF |
| 101 | LFLVGLELDL SSIRRSGKRA FGIAVAGITL PFIAGVGVAF |
| | VIRNTLYTAA |
| 151 | DKPGYAEFLV FMGVALSITA FPVLARILAE LKLLTTQIGE |
| | TAMAAAAFND |
| 201 | VAAWILLALA VALAGNGGEG GGEKKSPLVS LWVLLSGAGF |
| | VVFMLVVIRP |
| 251 | GMKWVAKRGS PENDVVRESY VCLTLAGVMV SGFATDLIGI |
| | HSIFGAFVFG |
| 301 | LTIPKDGEFG QRLIERIEDF VSGLLLPLYF ATSGLKTDVA |
| | KIRGAESWGM |
| 351 | LGLVVVTACA GKIVGTFVVA VMVKVPAREA LTLGFLMNTK |
| | GLVELIVLNI |
| 401 | GKEKKVLNDE TFAILVLMAL FTTFITTPTV MAIYKPARGT |
| | HRKLKDLSAS |
| 451 | QDSTKEELRI LACLHGPANV SSLISLVESI RTTKILRLKL |
| | FVMHLMELTE |
| 501 | RSSSIIMVQR ARKNGLPFVH RYRHGERHSN VIGGFEAYRQ |
| | LGRVAVRPIT |
| 551 | AVSPLPTMHE DICHMADTKR VTMIILPFHK RWNADHGHSH |
| | HHQDGGGDGN |
| 601 | VPENVGHGWR LVNQRVLKNA PCSVAVLVDR GLGSIEAQTL |
| | SLDGSNVVER |
| 651 | VCVIFFGGPD DRESIELGGR MAEHPAVKVT VIRFLVRETL |
| | RSTAVTLRPA |
| 701 | PSKGKEKNYA FLTTNVDPEK EKELDEGALE DFKSKWKEMV |
| | EYKEKEPNNI |
| 751 | IEEILSIGQS KDFDLIVVGR GRIPSAEVAA LAERQAEHPE |
| | LGPIGDVLAS |
| 801 | SINHIIPSIL VVQQHNKAHV EDITVSKIVS ESSLSINGDT |
| | NV |

### Example 3: Plant Materials, Growth Conditions, and Stress Treatments

Water-deficit stress treatments for Arabidopsis plants CHX20 transgenics were performed in parallel to all the genotypes by withholding water from three-week-old plants until reaching an average control soil weight of 45%, typically within 14 days. At the defined time points, plants' phenotypic responses to water deficit stress were measured.

Hybrid Poplar INRA 717-1B4, Cas9-mediated knockout, and overexpression lines of CHX20 were propagated in soil (Sungro Metro-mix 830). The pots were uniformly filled with the same amount of soil, ensuring consistency across all the pots. The survived stem cuttings that showed similar growth rates were grown for 3-4 months in the greenhouse under controlled conditions and were used for experiments. Pots were automatically watered by drip irrigation, maintaining daily field capacity. Water-deficit conditions were induced by withholding water on day 0 until plants completely wilted, between 3 and 10 days. Leaves were excised at defined time points, immediately frozen in liquid nitrogen, and stored at −80° C. until further use. At least three biological replicates per independent transgenic event were used in the experiments.

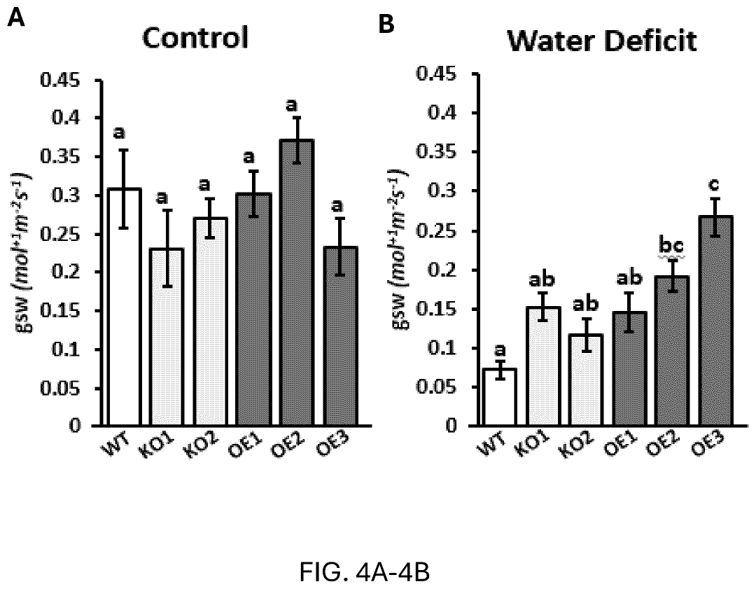

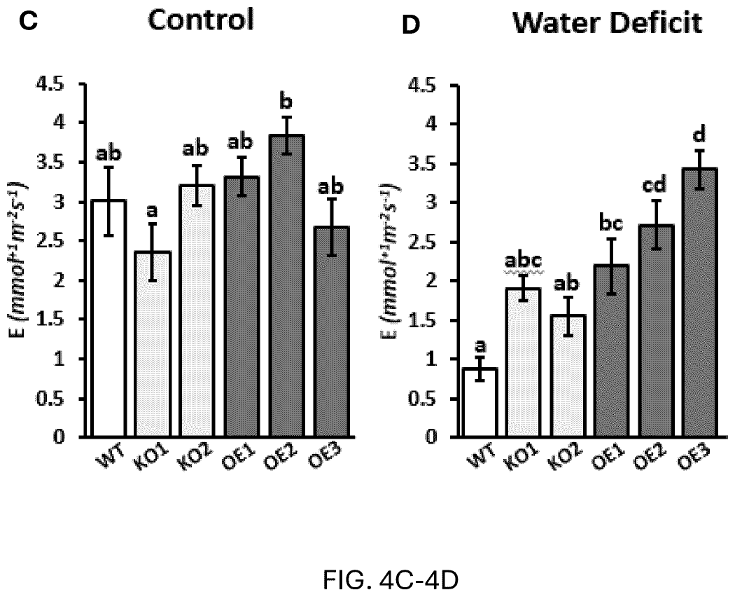

Overexpression of 30S was found to influence stomatal behavior and enhance stomatal opening under water deficit conditions. Under standard control growth conditions, no significant differences in photosynthetic parameters between knockout (KO) and overexpression (OE) lines of 30S. However, under water deficit conditions (14 days post cessation of watering), the 30S OE exhibited significantly higher stomatal conductance and transpiration rates, measured using a Licor 600 system, compared to wild-type (WT) plants and 30S KO (FIG. 4B). These findings suggest that overexpression of 30S enhances the tolerance of transgenic OE plants to water deficit stress by delaying the adverse effects of water limitation.

The overexpression of the 30S appears to optimize gas exchange during the initial phases of water deficit, while water remains available, subsequently transitioning to a more conservative approach as water deficit intensifies suggesting that 30S OE plants prioritize resource utilization, particularly carbon dioxide and light, during the early stages of stress. The overexpression of 30S may confer adaptive advantages, by regulating gas exchange and photosynthesis through modified stomatal responses enabling prolonged photosynthetic activity under water deficit conditions by delaying stomatal closure and maintaining carbon fixation. This mechanism supports sustained growth and energy production by preserving stomatal conductance and transpiration, thereby mitigating the early onset of drought-induced senescence. Moreover, the regulation of CHX20 by 30S ribosomal protein may enhance ion homeostasis and guard cell function, potentially triggering stress-responsive pathways that further improve the plant's adaptive response to water deficit conditions. These data indicate that 30S overexpression can delay the negative effects of water deficit stress, promoting metabolic processes critical to survival under water deficit stress.

1. A genetically modified plant, plant cell, or plant tissue, comprising an exogenous nucleic acid which comprises a nucleotide sequence encoding a 30S ribosomal protein or a homolog thereof, wherein the 30S ribosomal protein or the homolog thereof is expressed in the plant, plant cell, or plant tissue.

2. The genetically modified plant, plant cell, or plant tissue of claim 1, wherein the plant, plant cell, or plant tissue comprises an increase in expression of endogenous cation/H+ exchanger 20 (CHX20) gene or a homolog thereof in the plant, plant cell, or plant tissue as compared to CHX20 gene expression in a wild-type plant, plant cell, or plant tissue.

3. The genetically modified plant, plant cell, or plant tissue of claim 1, wherein the nucleotide sequence has at least 90% sequence identity to the nucleotide sequence shown in SEQ ID NO: 1.

4. The genetically modified plant, plant cell, or plant tissue of claim 1, wherein the 30S ribosomal protein or the homolog thereof has at least 90% sequence identity to the amino acid sequence shown in SEQ ID NO: 2.

5. The genetically modified plant, plant cell, or plant tissue according to claim 1, wherein the exogenous nucleic acid is stably integrated into the plant genome.

6. The genetically modified plant, plant cell, or plant tissue according claim 1, wherein the nucleotide sequence is operably linked to a heterologous promoter.

7. The genetically modified plant, plant cell, or plant tissue according to claim 1, wherein the heterologous promoter is a constitutive promoter, an inducible promoter, or a native promoter.

8. The genetically modified plant, plant cell, or plant tissue according to claim 1, wherein the heterologous promoter is a 35S promoter.

9. The genetically modified plant, plant cell, or plant tissue according to claim 1, wherein the plant is a monocot or a dicot.

10. The genetically modified plant, plant cell, or plant tissue according to claim 1, wherein the plant is selected from the group consisting of genera Acer, Afzelia, Allium, Arabidopsis, Agrostis, Avena, Betula, Brassica, Capsicum, Citrullus, Cucumis, Eucalyptus, Fagus, Festuca, Fraxinus, Fragaria, Glycine, Gossypium, Hordeum, Ipomoea, Jatropha, Juglans, Lemna, Lolium, Malus, Manihot, Medicago, Micropus, Milium, Miscanthus, Nicotiana, Oryza, Pennisetum, Phalaris, Phleum, Picea, Pinus, Poa, Populus, Prunus, Quercus, Rosa, Salix, Solanum, Sorghum, Spinacia, Tectona, Trifolium, Triticum, Panicum, Saccharum, Setaria, Zea, and Zoysia.

11. The genetically modified plant, plant cell, or plant tissue of claim 10, wherein the plant is Arabidopsis.

12. The genetically modified plant, plant cell, or plant tissue of claim 10, wherein the plant is Populus.

13. The genetically modified plant, plant cell, or plant tissue according to claim 1, wherein the plant displays one or more of the following characteristics:
retains water within its cell by altering its stomatal aperture;
has altered stomatal aperture length compared to a wild type plant;
has improved gaseous exchange, transpiration, and photosynthesis under severe water deficit conditions compared to a wild type plant;
has higher survival rate in water deficit conditions compared to a wild type plant; and
has delayed leaf senescence in drought conditions compared to a wild type plant.

14. The genetically modified plant, plant cell, or plant tissue of claim 13, wherein the drought condition is a cyclic drought condition or a short-term drought condition.

15. A method of improving drought tolerance and water loss in a plant, plant cell, or plant tissue comprising an exogenous nucleic acid sequence encoding a 30S ribosomal protein or a homolog thereof, wherein the 30S ribosomal protein or the homolog thereof is expressed in the plant, plant cell, or plant tissue.

16.-28. (canceled)

29. A genetically modified plant, plant cell, or plant tissue, comprising an exogenous nucleic acid which comprises a nucleotide sequence encoding a cation/H⁺ exchanger 20 (CHX20) protein or a homolog thereof, wherein the nucleotide sequence is operably linked to a heterologous promoter and the CHX20 protein or homolog thereof is expressed in the plant, plant cell, or plant tissue.

30.-40. (canceled)

41. A method of improving drought tolerance and water loss in a plant, plant cell, or plant tissue comprising an exogenous nucleic acid sequence encoding a cation/H⁺ exchanger 20 (CHX20) or a homolog thereof, wherein the CHX20 or the homolog thereof is expressed in the plant, plant cell, or plant tissue.

42.-53. (canceled)
